SEQUENCE LISTING (27 sequences)

SEQ ID NO: 1 - DNA, 3054 bp, Homo sapiens
atggttttctctgtcaggcagtgtggccatgttggcagaactgaagaagttttactgacgttcaagatattccttgtcatcatttgtcttcatgtcgttctggtaacatccctggaagaagatactgataattccagtttgtcaccaccacctgctaaattatctgttgtcagttttgccccctcctccaatggtactccagaggttgaaacaacaagcctcaatgatgttactttaagcttactcccttcaaacgaaacagaaaaaactaaaatcactatagtaaaaaccttcaatgcatcaggcgtcaaaccccagagaaatatctgcaatttgtcatctatttgcaatgactcagcattttttagaggtgagatcatgtttcaatatgataaagaaagcactgttccccagaatcaacatataacgaatggcaccttaactggagtcctgtctctaagtgaattaaaacgctcagagctcaacaaaaccctgcaaaccctaagtgagacttactttataatgtgtgctacagcagaggcccaaagcacattaaattgtacattcacaataaaactgaataatacaatgaatgcatgtgctgtaatagctgctttggaaagagtaaagattcgaccaatggaacactgctgctgttctgtcaggataccctgcccttcctccccagaagagttggaaaagcttcagtgtgacctgcaggatcccattgtctgtcttgctgaccatccacgtggcccaccattttcttccagccaatccatcccagtggtgcctcgggccactgtgctttcccaggtccccaaagctacctcttttgctgagcctccagattattcacctgtgacccacaatgttccctctccaataggggagattcaacccctttcaccccagccttcagctcccatagcttccagccctgccattgacatgcccccacagtctgaaacgatctcttcccctatgccccaaacccatgtctccggcaccccacctcctgtgaaagcctcattttcctctcccaccgtgtctgcccctgcgaatgtcaacactaccagcgcacctcctgtccagacagacatcgtcaacaccagcagtatttctgatcttgagaaccaagtgttgcagatggagaaggctctgtccttgggcagcctggagcctaacctcgcaggagaaatgatcaaccaagtcagcagactccttcattccccgcctgacatgctggcccctctggctcaaagattgctgaaagtagtggatgacattggcctacagctgaacttttcaaacacgactataagtctaacctccccttctttggctctggctgtgatcagagtgaatgccagtagtttcaacacaactacctttgtggcccaagaccctgcaaatcttcaggtttctctggaaacccaagctcctgagaacagtattggcacaattactcttccttcatcgctgatgaataatttaccagctcatgacatggagctagcttccagggttcagttcaatttttttgaaacacctgctttgtttcaggatccttccctggagaacctctctctgatcagctacgtcatatcatcgagtgttgcaaacctgaccgtcaggaacttgacaagaaacgtgacagtcacattaaagcacatcaacccgagccaggatgagttaacagtgagatgtgtattttgggacttgggcagaaatggtggcagaggaggctggtcagacaatggctgctctgtcaaagacaggagattgaatgaaaccatctgtacctgtagccatctaacaagcttcggcgttctgctggacctatctaggacatctgtgctgcctgctcaaatgatggctctgacgttcattacatatattggttgtgggctttcatcaatttttctgtcagtgactcttgtaacctacatagcttttgaaaagatccggagggattacccttccaaaatcctcatccagctgtgtgctgctctgcttctgctgaacctggtcttcctcctggactcgtggattgctctgtataagatgcaaggcctctgcatctcagtggctgtatttcttcattattttctcttggtctcattcacatggatgggcctagaagcattccatatgtacctggcccttgtcaaagtatttaatacttacatccgaaaatacatccttaaattctgcattgtcggttggggggtaccagctgtggttgtgaccatcatcctgactatatccccagataactatgggcttggatcctatgggaaattccccaatggttcaccggatgacttctgctggatcaacaacaatgcagtattctacattacggtggtgggatatttctgtgtgatatttttgctgaacgtcagcatgttcattgtggtcctggttcagctctgtcgaattaaaaagaagaagcaactgggagcccagcgaaaaaccagtattcaagacctcaggagtatcgctggccttacatttttactgggaataacttggggctttgccttctttgcctggggaccagttaacgtgaccttcatgtatctgtttgccatctttaataccttacaaggatttttcatattcatcttttactgtgtggccaaagaaaatgtcaggaagcaatggaggcggtatctttgttgtggaaagttacggctggctgaaaattctgactggagtaaaactgctactaatggtttaaagaagcagactgtaaaccaaggagtgtccagctcttcaaattccttacagtcaagcagtaactccactaactccaccacactgctagtgaataatgattgctcagtacacgcaagcgggaatggaaatgcttctacagagaggaatggggtctcttttagtgttcagaatggagatgtgtgccttcacgatttcactggaaaacagcacatgtttaacgagaaggaagattcctgcaatgggaaaggccgtatggctctcagaaggacttcaaagcggggaagcttacactttattgagcaaatgtga

SEQ ID NO: 2 - protein, 1017 aa, Homo sapiens
MVFSVRQCGHVGRTEEVLLTFKIFLVIICLHVVLVTSLEEDTDNSSLSPPPAKLSVVSFAPSSNGTPEVETTSLNDVTLSLLPSNETEKTKITIVKTFNASGVKPQRNICNLSSICNDSAFFRGEIMFQYDKESTVPQNQHITNGTLTGVLSLSELKRSELNKTLQTLSETYFIMCATAEAQSTLNCTFTIKLNNTMNACAVIAALERVKIRPMEHCCCSVRIPCPSSPEELEKLQCDLQDPIVCLADHPRGPPFSSSQSIPVVPRATVLSQVPKATSFAEPPDYSPVTHNVPSPIGEIQPLSPQPSAPIASSPAIDMPPQSETISSPMPQTHVSGTPPPVKASFSSPTVSAPANVNTTSAPPVQTDIVNTSSISDLENQVLQMEKALSLGSLEPNLAGEMINQVSRLLHSPPDMLAPLAQRLLKVVDDIGLQLNFSNTTISLTSPSLALAVIRVNASSFNTTTFVAQDPANLQVSLETQAPENSIGTITLPSSLMNNLPAHDMELASRVQFNFFETPALFQDPSLENLSLISYVISSSVANLTVRNLTRNVTVTLKHINPSQDELTVRCVFWDLGRNGGRGGWSDNGCSVKDRRLNETICTCSHLTSFGVLLDLSRTSVLPAQMMALTFITYIGCGLSSIFLSVTLVTYIAFEKIRRDYPSKILIQLCAALLLLNLVFLLDSWIALYKMQGLCISVAVFLHYFLLVSFTWMGLEAFHMYLALVKVFNTYIRKYILKFCIVGWGVPAVVVTIILTISPDNYGLGSYGKFPNGSPDDFCWINNNAVFYITVVGYFCVIFLLNVSMFIVVLVQLCRIKKKKQLGAQRKTSIQDLRSIAGLTFLLGITWGFAFFAWGPVNVTFMYLFAIFNTLQGFFIFIFYCVAKENVRKQWRRYLCCGKLRLAENSDWSKTATNGLKKQTVNQGVSSSSNSLQSSSNSTNSTTLLVNNDCSVHASGNGNASTERNGVSFSVQNGDVCLHDFTGKQHMFNEKEDSCNGKGRMALRRTSKRGSLHFIEQM

SEQ ID NO: 3 - protein, 15 aa, artificial sequence
  peptide
TSFGVLLDLSRTSVL

SEQ ID NO: 4 - DNA, 35 bp, artificial sequence
  primer
tttaaacttaaggccatggttttctctgtcaggca

SEQ ID NO: 5 - DNA, 35 bp, artificial sequence
  primer
cgagcggccgcttacatttgctcaataaagtgtaa

SEQ ID NO: 6 - DNA, 18 bp, Artificial sequence
  primer
acaagcttcggcgttctg

SEQ ID NO: 7 - DNA, 18 bp, Artificial sequence
  primer
cgatccagcgtaatctgg

SEQ ID NO: 8 - DNA, 339 bp, Mus sp.
gatgtgcagcttcaggagtcgggacctggcctggtgaaaccttctcagtctctgtccctcacctgcactgtcactggctactcaatcaccagtgattatgcctggaactggatccggcagtttccaggaaacaaactggagtggctgggctacataagcttcaatgataacactaactacaacccatctctcaaaagtcgaatctctatcactcgagacacatccaagaaccagttcttcctgcagttgaattctgtgactactgaggacacagccacatattactgtacaaggagggtggactactggggtcaaggaacctcagtcaccgtctcctca

SEQ ID NO: 9 - DNA, 336 bp, Mus sp.
gatgttgtgatgacccaaactccactctccctgcctgtcagtcttggagatcaagcctccatctcttgcagatctagtcagagccttgtacacagtaatggaaacaactatttacattggtatttgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtggacgttcggtggaggcaccaagctggaaatcaaa

SEQ ID NO: 10 - protein, 113 aa, Mus sp.
DVQLQESGPGLVKPSQSLSLTCTVTGYSITSDYAWNWIRQFPGNKLEWLGYISFNDNTNYNPSLKSRISITRDTSKNQFFLQLNSVTTEDTATYYCTRRVDYWGQGTSVTVSS

SEQ ID NO: 11 - protein, 112 aa, Mus sp.
DVVMTQTPLSLPVSLGDQASISCRSSQSLVHSNGNNYLHWYLQKPGQSPKLLIYKVSNRFSGVPDRFSGSGSGTDFTLKISRVEAEDLGVYFCSQSTHVPWTFGGGTKLEIK

SEQ ID NO: 12 - DNA, 360 bp, Mus sp.
caggttactctgaaagagtctggccctgggatattgcagccctcccagaccctcagtctgacttgttctttctctgggttttcactgagcacttctggtgtgggtgtgagctggattcgtcagccttcaggaaagggtctggagtggctggcacacatttactgggatgatgataagcgctataacccatccctgaagagccggctcacaatctccaaggatacctccagaaaccaggtattcctcaagatcaccagtgtggacactgcagatactgccacatactactgtgctcgaagagtattcattattacggcctttgactactggggccaaggcaccactctcacagtctcctca

SEQ ID NO: 13 - DNA, 321 bp, Mus sp.
gatatccagatgacacagactacatcctccctgtctgcctctctgggagacagagtcaccatcagttgcagggcaagtcaggacattagcaattacttaaactggtatcagcagaaaccagatggaactgttaaactcctgatctactacacatcaaacttacactcaggagtcccatcaaggttcagtggcagtgggtctggagcagattattctctcaccattggcaacctggagcaagaagatattgccacttacttttgccaacagggtaatacgcttccttggacgttcggtggaggcaccaagctggaaatcaaa

SEQ ID NO: 14 - protein, 120 aa, Mus sp.
QVTLKESGPGILQPSQTLSLTCSFSGFSLSTSGVGVSWIRQPSGKGLEWLAHIYWDDDKRYNPSLKSRLTISKDTSRNQVFLKITSVDTADTATYYCARRVFIITAFDYWGQGTTLTVSS

SEQ ID NO: 15 - protein, 107 aa, Mus sp.
DIQMTQTTSSLSASLGDRVTISCRASQDISNYLNWYQQKPDGTVKLLIYYTSNLHSGVPSRFSGSGSGADYSLTIGNLEQEDIATYFCQQGNTLPWTFGGGTKLEIK

SEQ ID NO: 16 - DNA, 366 bp, Mus sp.
caggtttctctgaaagagtctggccctgggatattgcagccctcccagaccctcagtctgacttgttctttctctgggttttcactgagcacttctggtatgggtgtgagctggattcgtcagccttcaggaaagggtctggagtggctggcacacatttactgggatgatgacaagcgctataacccatccctgaagagccggctcacaatctccaaggatacctccagcaacctggtattcctcaagatcaccagtgtggacactgcagatactgccacatactactgtgctcgaagggaagtacgacgtgattactatgctatggactactggggtcaaggaacctcagtcaccgtctcctca

SEQ ID NO: 17 - DNA, 324 bp, Mus sp.
agtattgtgatgacccagactcccaaattcctgcttgtctcagcaggagacaggattaccatagcctgcagggccagtcagagtgtgagtaatgatgtagcttggtaccaacagaagccagggcagtctcctaaactgctgataaactatacatccaatcgctacactggagtccctgatcgcttcactggcagtggatatgggacggatttcactttcaccatcagcactgtgcaggctgaagacctggcagtttatttctgtcagcaggcttatagctctccgtggacgttcggtggaggcaccaagctggaaatcaaacgg

SEQ ID NO: 18 - protein, 122 aa, Mus sp.
QVSLKESGPGILQPSQTLSLTCSFSGFSLSTSGMGVSWIRQPSGKGLEWLAHIYWDDDKRYNPSLKSRLTISKDTSSNLVFLKITSVDTADTATYYCARREVRRDYYAMDYWGQGTSVTVSS

SEQ ID NO: 19 - protein, 107 aa, Mus sp.
SIVMTQTPKFLLVSAGDRITIACRASQSVSNDVAWYQQKPGQSPKLLINYTSNRYTGVPDRFTGSGYGTDFTFTISTVQAEDLAVYFCQQAYSSPWTFGGGTKLEIK

SEQ ID NO: 20 - DNA, 339 bp, Mus sp.
gatgtgcagcttcaggagtcgggacctggcctggtgaaaccttctcagtctctgtccctcacctgcactgtcactggctactcaatcaccagtgattatgcctggaactggatccggcagtttccaggaaacaaactggagtggatgggctacataagctacagtgattacactagctacaacccatctctcaaaagtcgaatctctatcactcgagacacatccaagaaccagttcttcctgcagttgaattctgtgactactgaggacacagccacatattactgtgcaagaagggtggactactggggtcaaggaacctcagtcaccgtctcctca

SEQ ID NO: 21 - DNA, 336 bp, Mus sp.
gatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtggacgttcggtggaggcaccacgctggaaatcaaa

SEQ ID NO: 22 - protein, 113 aa, Mus sp.
DVQLQESGPGLVKPSQSLSLTCTVTGYSITSDYAWNWIRQFPGNKLEWMGYISYSDYTSYNPSLKSRISITRDTSKNQFFLQLNSVTTEDTATYYCARRVDYWGQGTSVTVSS

SEQ ID NO: 23 - protein, 112 aa, Mus sp.
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPWTFGGGTTLEIK

SEQ ID NO: 24 - DNA, 339 bp, Mus sp.
gatgtgcagcttcaggagtcgggacctggcctggtgaaaccttctcagtctctgtccctcacctgcactgtcactggctactcaatcaccagtgattatgcctggaactggatccggcagtttccaggaaacaaactggagtggatgggctacataagcttcagtgatagcactagctacaacccatctctcaaaagtcgaatctctatcactcgagacacatccaagaaccagttcttcctgcagttgaattctgtgactactgaggacacagccacatattactgtgcaagaaggggggactactggggtcaaggaacctcagtcaccgtctcctca

SEQ ID NO: 25 - DNA, 336 bp, Mus sp.
gatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatcttccgtggacgttcggtggaggcaccaagctggaaatcaaa

SEQ ID NO: 26 - protein, 113 aa, Mus sp.
DVQLQESGPGLVKPSQSLSLTCTVTGYSITSDYAWNWIRQFPGNKLEWMGYISFSDSTSYNPSLKSRISITRDTSKNQFFLQLNSVTTEDTATYYCARRGDYWGQGTSVTVSS

SEQ ID NO: 27 - protein, 112 aa, Mus sp.
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHLPWTFGGGTKLEIK

(19) United States
(12) Patent Application Publication
(10) Pub. No.: US 2017/0333522 A1
(43) Pub. Date: Nov. 23, 2017

(54) EXPRESSION AND FUNCTION OF GPR64/ADGRG2 IN ENDOCRINE SYSTEMS AND METHODS TO TARGET IT THERAPEUTICALLY

(71) Applicants: Nariman Balenga, Bethesda, MD (US); John Olson, Baltimore, MD (US)

(72) Inventors: Nariman Balenga, Bethesda, MD (US); John Olson, Baltimore, MD (US)

(21) Appl. No.: 15/589,526

(22) Filed: May 8, 2017

Related U.S. Application Data
(60) Provisional application No. 62/333,261, filed on May 8, 2016.

(51) Int. Cl. A61K 38/17; A61K 45/06; G01N 33/50; C12Q 1/68; A61K 48/00

(52) U.S. Cl. CPC A61K 38/177; G01N 33/5023; C12Q 1/6883; A61K 45/06; G01N 33/5041; A61K 48/00; C12Q 2600/158; C12Q 2600/136; G01N 2333/635; G01N 2333/726; G01N 2800/04

(57) ABSTRACT

Provided herein are methods of treating hypercalcemia in a subject, comprising administering to the subject a therapeutically effective amount of a composition that decreases the expression level and/or activity of GPR64. Compositions comprising the therapeutic agents and methods of screening for agonists and inhibitors of GPR64 are also provided. Compositions and methods for treating primary hyperparathyroidism, secondary hyperparathyroidism, hypoparathyroidism, and osteoporosis without hyperparathyroidism are also provided.

### CROSS REFERENCE TO RELATED APPLICATIONS

This application claims the benefit of U.S. Provisional Appl. No. 62/333,261, filed May 8, 2016. The contents of the aforesaid application are incorporated by reference in their entirety.

### INCORPORATION-BY-REFERENCE OF MATERIAL SUBMITTED ELECTRONICALLY

Incorporated by reference in its entirety herein is a computer-readable sequence listing submitted concurrently herewith and identified as follows: One 29, 755 Byte ASCII (Text) file named “sequence_listing_ST25.txt,” created on May 3, 2017.

### FIELD OF THE INVENTION

The field of the invention generally relates to medicine and pharmaceuticals. In particular, the field of the invention relates to compositions and methods for treating hypercalcemia.

### BACKGROUND

Primary hyperparathyroidism (PHPT) is a common endocrine disorder, caused by parathyroid gland neoplasia that variably causes morbidity of the renal, musculoskeletal, cardiovascular, and neural systems. Brown E M., Subcell Biochem. 2007; 45:139-67. In all cases of PHPT, the neoplastic parathyroid gland(s) hypersecrete parathyroid hormone (PTH), leading to absolute or relative hypercalcemia. Brown E M., Am J Med. 1999; 106(2):238-53. The underlying mechanism(s) of PTH hypersecretion are incompletely understood; however, most experimental evidence supports impaired sensing of extracellular Ca²⁺ concentrations by the calcium-sensing receptor (CaSR), a class C G protein-coupled receptor (GPCR) expressed on the surface of parathyroid chief cells, and the consequent impaired negative feedback to PTH secretion. Zhang et al., Sci China Life Sci. 2015; 58(1):14-27. Normally, the CaSR signals through Gαq and Gαi heterotrimeric G proteins to increase the intracellular Ca²⁺-mediated cytoskeletal barrier (Shoback et al., Proc Natl Acad Sci USA. 1984; 81(10):3113-7) and to reduce cAMP-mediated fusion of secretory granules to the plasma membrane (Brown et al., Proc Natl Acad Sci USA. 1977; 74(10):4210-3), which ultimately suppress PTH secretion by the parathyroids. Several mechanisms of abnormal CaSR biology including aberrant expression of negative regulators of CaSR (Koh et al., Mol Endocrinol. 2011; 25(5):867-76), inactivating mutations (Egbuna et al., Best Pract Res Clin Rheumatol. 2008; 22(1):129-48) or reduced expression (Farnebo et al., J Clin Endocrinol Metab 1997; 82(10):3481-6) of CaSR lead to PHPT. These abnormalities are not universally seen, leading to the notion that other mechanism(s) of CaSR dysregulation are involved in the pathogenesis of PHPT.

This background information is provided for informational purposes only. No admission is necessarily intended, nor should it be construed, that any of the preceding information constitutes prior art against the present invention.

### SUMMARY

It is to be understood that both the foregoing general description of the embodiments and the following detailed description are exemplary, and thus do not restrict the scope of the embodiments.

It is reported herein that the orphan adhesion G protein-coupled receptor G2, GPR64/ADGRG2, is significantly upregulated in parathyroid tumors collected from patients with PHPT, and that GPR64 antagonizes calcium-stimulated signaling by CaSR. It is also demonstrated that activation of GPR64 in primary human parathyroid cells results in elevated secretion of PTH. Furthermore, it is shown that a constitutively active mutant of GPR64 or a peptide-activated GPR64 blunts CaSR-mediated cAMP suppression in a HEK-CaSR model system. These results demonstrate that GPR64 can antagonize CaSR-mediated inactivation of adenylate cyclase/PTH secretion and suggest that this orphan GPCR is involved in the pathogenesis of PHPT.

In one aspect, the invention provides a method of treating hypercalcemia in a subject, comprising administering to the subject a therapeutically effective amount of a composition that decreases the level and/or activity of GPR64.

In another aspect, the invention provides a composition for treating hypercalcemia comprising an effective amount of an agent that is capable of decreasing the level and/or activity of GPR64 and a pharmaceutically acceptable carrier.

In another aspect, the invention provides a method of screening for an agent which modulates the expression level or activity of GPR64 comprising:
i) contacting cells expressing GPR64 with the agent; and
ii) assaying the agent's effect on the expression level or activity of GPR64.

It is to be understood that both the foregoing general description and the following detailed description are exemplary, and thus do not restrict the scope of the invention. Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The skilled artisan will understand that the drawings, described below, are for illustration purposes only. The drawings are not intended to limit the scope of the present teachings in any way.

FIG. 1. (A) Human parathyroid sections from healthy subject glands or adenomas and normal glands of PHPT patients were stained with an antibody targeted to the N-terminal segment of GPR64 (brown) followed by counterstaining with hematoxylin (purple). Neighboring sections were stained with hematoxylin and eosin. Arrows show expression at the cell-cell contacts. Scale bars: 100 μm; original magnification: 400×. (B) Comparison of GPR64 staining between parathyroids of healthy controls (n=6) and adenomas of PHPT patients (n=12). *: P=0.0315 Mann-Whitney U test. (C) Comparison of GPR64 staining between parathyroid adenomas and normal glands of same PHPT patients (n=6). (D) Freshly isolated parathyroid adenoma cells were incubated with rabbit anti-GPR64 and mouse anti-CaSR antibodies followed by staining with AlexaFluor-594 conjugated goat anti-rabbit (red) and AlexaFluor-488 conjugated goat anti-mouse antibody and counterstained with DAPI (nuclear). Representative images from 4 donors are shown. Scale bar: 20 μm; original magnification: 400×.

FIG. 2. (A) HEK cells were transfected with pCRE-Luc in combination with 200 ng of pcDNA3.1, GPR64 or GPR64ΔNTF plasmids and were stimulated with increasing concentrations of 15 amino acid-long human GPR64 peptide (P-15) for 5 hrs. Luciferase activity (CRE induction) was measured and data were normalized to the values of cells transfected with pcDNA3.1. Data are mean±SD from 3-4 independent experiments conducted in triplicates. (B) Forty eight hrs post-transfection with pcDNA3.1, GPR64 or GPR64ΔNTF plasmids, HEK cells were stimulated with DMSO (vehicle) or 100 μM P-15 peptide for 30 min and cAMP accumulation was measured in an ELISA assay at 405 nm. Data were corrected to the protein concentration in samples. Data are mean±SD from 3 independent experiments conducted in triplicates. *: P<0.05, ****: P<0.0001. (C) Freshly isolated human adenoma parathyroid cells were stimulated with increasing concentrations of Ca²⁺ and P-15 or DMSO (vehicle) for 30 min and the concentration of secreted intact PTH was measured by ELISA. Data were normalized to the PTH secretion in vehicle-treated cells cultured in 0.5 mM Ca²⁺. Data are mean±SD, representative of 5 independent experiments. **: P<0.01, ***: P<0.001, ****: P<0.0001. (D) Freshly isolated human adenoma parathyroid cells were stimulated with increasing concentrations of Ca²⁺ and forskolin (FSK, 3 and 5 μM) or DMSO (vehicle) for 30 min and the concentration of secreted intact PTH was measured by ELISA. Data were normalized to the PTH secretion in vehicle-treated cells cultured in 0.5 mM Ca²⁺. Data are mean±SD representative of 3 independent experiments. *: P<0.05, ***: P<0.001. (E) HEK-CaSR cells were stimulated with 1.25 or 3 mM Ca²⁺ for 30 min, followed by an additional 30 min incubation with either 10 μM forskolin (FSK) or DMSO (vehicle). cAMP accumulation was measured as in panel B. Data are mean±SD representing 3 independent experiments conducted in duplicates *: P<0.05. (F) Forty eight hrs post-transfection with pcDNA3.1, GPR64 or GPR64ΔNTF, HEK-CaSR cells were stimulated with either 1.25 mM or 3 mM Ca²⁺ followed by an additional 30 min incubation with either 100 μM P-15 or DMSO (vehicle). cAMP accumulation was measured as in panel B. Data are mean±SD representing 3 independent experiments conducted in duplicates. *: P<0.05, **: P<0.01, ***: P<0.001.

FIG. 3. (A) Human parathyroid sections from adenomas of PHPT patients were stained with rabbit IgG control antibody and (B) human heart sections were stained with rabbit anti-GPR64 antibody followed by counterstaining with hematoxylin (purple). Neighboring sections were stained with hematoxylin and eosin. Representative images from 3 donors are shown. Scale bars: 100 μm; original magnification: 400×.

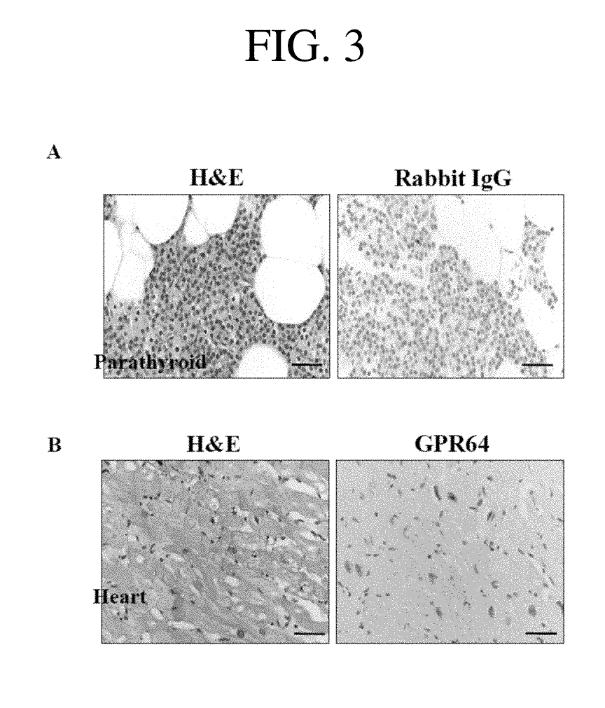

FIG. 4. Freshly isolated parathyroid adenoma cells were incubated with rabbit IgG and mouse IgG followed by staining with AlexaFluor-594 conjugated goat anti-rabbit and AlexaFluor-488 conjugated goat anti-mouse antibody and counterstained with DAPI (nuclear). Representative images from 3 donors are shown. Scale bar: 20 μm; original magnification: 400×.

FIG. 5. HEK cells transfected with pcDNA3.1, GPR64 or GPR64ΔNTF were fixed, incubated with rabbit anti-GPR64 antibody and stained with AlexaFluor-594 conjugated goat anti-rabbit antibody and counterstained with DAPI (nuclear). Representative images from 3 independent experiments are shown. Scale bar: 50 μm; original magnification: 400×.

FIG. 6. HEK cells were transfected with pCRE-Luc plasmid along with increasing doses of pcDNA3.1, GPR64 or GPR64ΔNTF. Basal luciferase activity (CRE induction) was measured and data were normalized to the values of cells transfected with 50 ng pcDNA3.1. Data are mean±SD from 3-4 independent experiments conducted in triplicates. *: P<0.05, **: P<0.01, ****: P<0.0001.

FIG. 7. HEK cells were transfected with pCRE-Luc and GPR64 and were preincubated with DMSO (vehicle), H-89 (PKA inhibitor, 20 μM) or U0126 (MEK inhibitor, 10 μM) for 1 hr before stimulation with either DMSO or 100 μM P-15 peptide for 5 hrs. Luciferase assay data are normalized to vehicle-preincubated, DMSO stimulated cells. Data are mean±SD from 3 independent experiments conducted in triplicates. *: P<0.05, **: P<0.01.

FIG. 8. Surface expression of FLAG-tagged CaSR was measured by quantitative ELISA 48 hrs post-transfection with pcDNA3.1, GPR64 or GPR64ΔNTF and data were normalized to the values of empty vector-transfected cells. Data are mean±SD from 3 independent experiments conducted in quadruplicate.

FIG. 9. (A) Nucleotide sequence of antibody variable regions directed against GPR64. CDR sequences are shown bolded and underlined. GPR64-1 heavy chain variable region nucleotide sequence. (B) Nucleotide sequence of antibody variable regions directed against GPR64. CDR sequences are shown bolded and underlined. GPR64-1 light chain variable region nucleotide sequence. (C) Amino acid sequence of antibody variable regions directed against GPR64. CDR sequences are shown bolded and underlined. GPR64-1 heavy chain variable region amino acid sequence. (D) Amino acid sequence of antibody variable regions directed against GPR64. CDR sequences are shown bolded and underlined. GPR64-1 light chain variable region amino acid sequence.

FIG. 10. (A) Nucleotide sequence of antibody variable regions directed against GPR64. CDR sequences are shown bolded and underlined. GPR64-16 heavy chain variable region nucleotide sequence. (B) Nucleotide sequence of antibody variable regions directed against GPR64. CDR sequences are shown bolded and underlined. GPR64-16 light chain variable region nucleotide sequence. (C) Amino acid sequence of antibody variable regions directed against GPR64. CDR sequences are shown bolded and underlined. GPR64-1 heavy chain variable region amino acid sequence. (D) Amino acid sequence of antibody variable regions directed against GPR64. CDR sequences are shown bolded and underlined. GPR64-16 light chain variable region amino acid sequence.

FIG. 11. (A) Nucleotide sequence of antibody variable regions directed against GPR64. CDR sequences are shown bolded and underlined. GPR64-18 heavy chain variable region nucleotide sequence. (B) Nucleotide sequence of antibody variable regions directed against GPR64. CDR sequences are shown bolded and underlined. GPR64-18 light chain variable region nucleotide sequence. (C) Amino acid sequence of antibody variable regions directed against GPR64. CDR sequences are shown bolded and underlined. GPR64-18 heavy chain variable region amino acid sequence. (D) Amino acid sequence of antibody variable regions directed against GPR64. CDR sequences are shown bolded and underlined. GPR64-18 light chain variable region amino acid sequence.

FIG. 12. (A) Nucleotide sequences of antibody variable regions directed against GPR64. CDR sequences are shown bolded and underlined. GPR64-20 heavy chain variable region nucleotide sequence. (B) Nucleotide sequences of antibody variable regions directed against GPR64. CDR sequences are shown bolded and underlined. GPR64-20 light chain variable region nucleotide sequence. (C) Amino acid sequence of antibody variable regions directed against GPR64. CDR sequences are shown bolded and underlined. GPR64-20 heavy chain variable region amino acid sequence. (D) Amino acid sequence of antibody variable regions directed against GPR64. CDR sequences are shown bolded and underlined. GPR64-20 light chain variable region amino acid sequence.

FIG. 13. (A) Nucleotide sequences of antibody variable regions directed against GPR64. CDR sequences are shown bolded and underlined. GPR64-48 heavy chain variable region nucleotide sequence. (B) Nucleotide sequences of antibody variable regions directed against GPR64. CDR sequences are shown bolded and underlined. GPR64-48 light chain variable region nucleotide sequence. (C) Amino acid sequence of antibody variable regions directed against GPR64. CDR sequences are shown bolded and underlined. GPR64-48 heavy chain variable region amino acid sequence. (D) Amino acid sequence of antibody variable regions directed against GPR64. CDR sequences are shown bolded and underlined. GPR64-48 light chain variable region amino acid sequence.

FIG. 14A. Tertiary hyperparathyroidism following kidney transplantation. A patient had a kidney transplantation followed by the emergence of 3-4 parathyroid tumors and high PTH secretion. Isolated tumor cells from tertiary hyperparathyroidism gland A were stimulated with calcium (CaSR agonist) and P-15 peptide (GPR64 agonist) in vitro and PTH secretion was measured. At almost all concentrations of calcium, P-15 leads to higher PTH secretion.

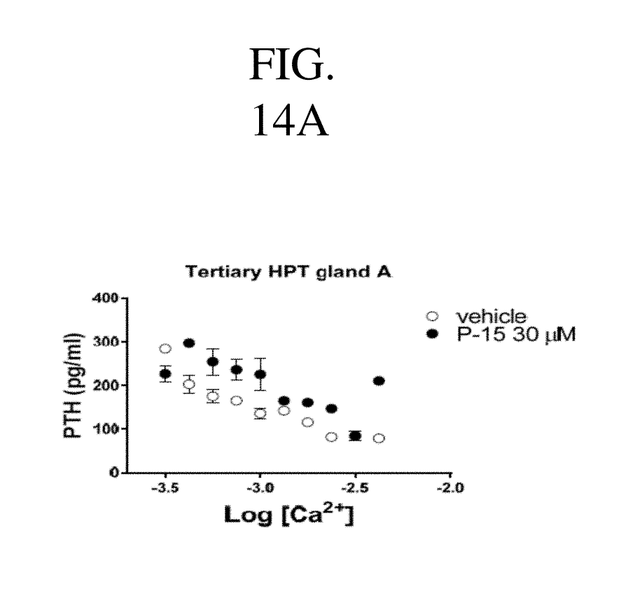

FIG. 14B. Tertiary hyperparathyroidism following kidney transplantation. A patient had a kidney transplantation followed by the emergence of 3-4 parathyroid tumors and high PTH secretion. Isolated tumor cells from tertiary hyperparathyroidism gland C were stimulated with calcium (CaSR agonist) and P-15 peptide (GPR64 agonist) in vitro and PTH secretion was measured. Cells express very high levels of GPR64 and are unresponsive to calcium-mediated suppression of PTH secretion.

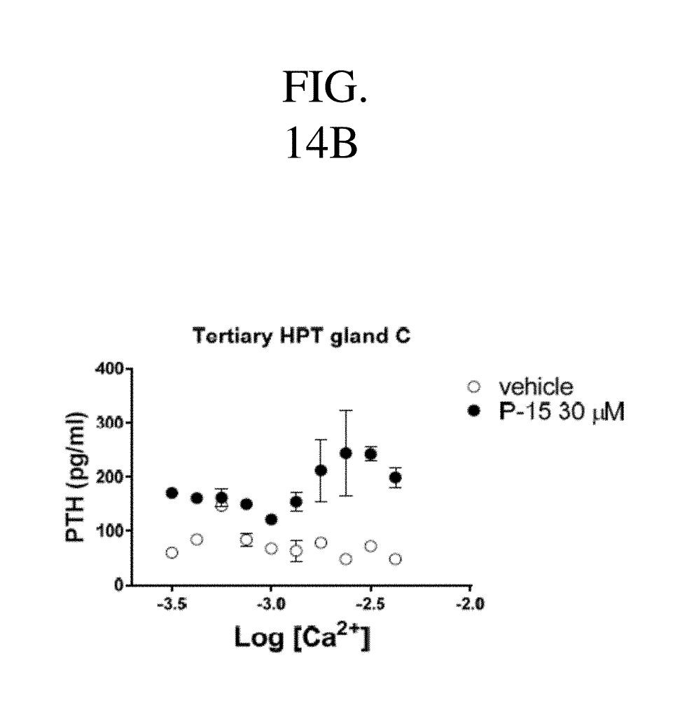

### DETAILED DESCRIPTION

G-protein Coupled Receptors (GPCRs) are expressed on surface of all human cell types and play multitude of physiological roles in health and diseases. Currently, more than 30% of drugs in the market target GPCRs. This invention shows that an orphan adhesion GPCR, GPR64, is expressed in the parathyroid glands of healthy humans and its expression is significantly elevated in patients suffering from dysregulated parathyroid hormone (PTH) secretion and patients with high serum calcium. In addition, without being bound by theory, this invention provides the cellular and molecular mechanisms by which GPR64 modulates PTH secretion. This invention shows that GPR64 antagonizes the calcium-sensing receptor (CASR) by various mechanisms including increasing the cyclic AMP levels. Therefore, small molecules or biologics that inhibit GPR64 or suppress its expression are regarded as interventions for treatment of patients with dysregulated calcium homeostasis. Also, this invention provides the methods by which small molecules or biologics can be screened for such modulatory effect. It is known that GPR64 is expressed in testis and plays a major role in male fertility. Therefore, the methods provided herein for screening of GPR64 agonists/modulators can be used to discover compounds as that can be useful to promote fertility in males.

Reference will now be made in detail to embodiments of the invention which, together with the drawings and the following examples, serve to explain the principles of the invention. These embodiments describe in sufficient detail to enable those skilled in the art to practice the invention, and it is understood that other embodiments may be utilized, and that structural, biological, and chemical changes may be made without departing from the spirit and scope of the present invention. Unless defined otherwise, all technical and scientific terms used herein have the same meanings as commonly understood by one of ordinary skill in the art.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. Furthermore, where the description of one or more embodiments uses the term “comprising,” those skilled in the art would understand that, in some specific instances, the embodiment or embodiments can be alternatively described using the language “consisting essentially of” and/or “consisting of.” As used herein, the term “about” means at most plus or minus 10% of the numerical value of the number with which it is being used.

It is contemplated that any method or composition described herein can be implemented with respect to any other method or composition described herein.

One skilled in the art may refer to general reference texts for detailed descriptions of known techniques discussed herein or equivalent techniques. These texts include Current Protocols in Molecular Biology (Ausubel et. al., eds. John Wiley & Sons, N.Y. and supplements thereto), Current Protocols in Immunology (Coligan et al., eds., John Wiley St Sons, N.Y. and supplements thereto), Current Protocols in Pharmacology (Enna et al., eds. John Wiley & Sons, N.Y. and supplements thereto) and Remington: The Science and Practice of Pharmacy (Lippincott Williams & Wilicins, 2Vt edition (2005)), for example.

### I. Therapeutic Methods

In one embodiment, the invention provides a method of treating hypercalcemia in a subject, comprising administering to the subject a therapeutically effective amount of a composition that decreases the level and/or activity of GPR64. In some embodiments, the hypercalcemia is caused by increased secretion of parathyroid hormone.

In some embodiments, the increased secretion of parathyroid hormone is caused by primary hyperparathyroidism (PHPT). In some embodiments, the primary hyperparathyroidism is caused by parathyroid adenoma, parathyroid hyperplasia, and parathyroid carcinoma, multiple endocrine neoplasia type 1, multiple endocrine neoplasia type 2A, and familial hyperparathyroidism.

In some embodiments, the invention provides a method of decreasing secretion of parathyroid hormone in a subject, comprising administering to the subject a therapeutically effective amount of a composition that decreases the level and/or activity of GPR64.

In some embodiments, the invention provides a method of increasing secretion of parathyroid hormone in a subject, comprising administering to the subject a therapeutically effective amount of an agonist or activator of GPR64. In some embodiments, the agonist or activator is a peptide (P-15) comprising the sequence TSFGVLLDLSRTSVL (SEQ ID NO:3), or a biologically active fragment or derivative thereof. In some embodiments, the invention provides a method of increasing secretion of parathyroid hormone in a subject, comprising administering to the subject a therapeutically effective amount of a vector encoding GPR64 or a biologically active fragment or derivative thereof. In some embodiments, the subject is administered an effective amount of a combination of an agonist or activator of GPR64 and a vector encoding GPR64 or a biologically active fragment or derivative thereof.

In some embodiments, the invention provides a method of treating secondary hyperparathyroidism (SHPT) in a subject, comprising administering to the subject a therapeutically effective amount of a composition that decreases the level and/or activity of GPR64. SHPT occurs mostly due to renal failure to excrete phosphate. Increased phosphate precipitates calcium in blood (hypocalcemia) and forces the parathyroid gland to secret more PTH, which finally leads to joint and bone pain.

In some embodiments, the hypercalcemia is caused by tertiary hyperparathyroidism. In some embodiments, the tertiary hyperparathyroidism follows kidney transplantation.

In some embodiments, the invention provides a method of treating hypoparathyroidism in a subject, comprising administering to the subject a therapeutically effective amount of an agonist or activator of GPR64. In some embodiments, the agonist or activator is a peptide (P-15) comprising the sequence TSFGVLLDLSRTSVL (SEQ ID NO:3), or a biologically active fragment or derivative thereof. In another embodiment, the invention provides a method of treating hypoparathyroidism in a subject, comprising administering to the subject a therapeutically effective amount of a vector encoding GPR64 or a biologically active fragment or derivative thereof. In some embodiments, the subject is administered an effective amount of a combination of an agonist or activator of GPR64, such as P-15 and a vector encoding GPR64 or a biologically active fragment or derivative thereof.

In some embodiments, the invention provides a method of treating osteoporosis in a subject, wherein the subject is without hyperparathyroidism, comprising administering to the subject a therapeutically effective amount of an agonist or activator of GPR64. If a subject has osteoporosis without hyperparathyroidism, activation of GPR64 will increase PTH secretion, which is believed to treat the osteoporosis. In some embodiments, the agonist or activator is a peptide (P-15) comprising the sequence TSFGVLLDLSRTSVL (SEQ ID NO:3), or a biologically active fragment or derivative thereof. In another embodiment, the invention provides a method of treating osteoporosis without hyperparathyroidism in a subject, comprising administering to the subject a therapeutically effective amount of a vector encoding GPR64 or a biologically active fragment or derivative thereof. In some embodiments, the subject is administered an effective amount of a combination of an agonist or activator of GPR64, such as P-15 and a vector encoding GPR64 or a biologically active fragment or derivative thereof.

The term “subject” as used herein is not limiting and is used interchangeably with patient. In some embodiments, the subject refers to animals, such as mammals. For example, mammals contemplated include humans, primates, dogs, cats, sheep, cattle, goats, pigs, horses, chickens, mice, rats, rabbits, guinea pigs, and the like.

As used herein, “treat” and all its forms and tenses (including, for example, treating, treated, and treatment) can refer to therapeutic or prophylactic treatment. In certain aspects of the invention, those in need of treatment include those already with a pathological condition of the invention (including, for example, hypercalcemia), in which case treating refers to administering to a subject (including, for example, a human or other mammal in need of treatment) a therapeutically effective amount of a composition so that the subject has an improvement in a sign or symptom of a pathological condition of the invention. The improvement can be any observable or measurable improvement. Thus, one of skill in the art realizes that a treatment can improve the patient's condition, but may not be a complete cure of the pathological condition. In other aspects of the invention, those in need of treatment include those in which a pathological condition is to be prevented, in which case treating refers to administering a therapeutically effective amount of a composition to a subject at risk of developing a disease or condition such as primary hyperparathyroidism.

In accordance with the invention, a “therapeutically effective amount” or “effective amount” is administered to the subject. As used herein a “therapeutically effective amount” or “effective amount” is an amount sufficient to decrease, suppress, or ameliorate one or more symptoms associated with the disease or condition.

In some embodiments, the composition useful in the methods of the invention comprises a nucleic acid molecule that comprises a nucleotide sequence that binds to at least a portion of a nucleotide sequence of GPR64. The nucleic acid molecule can be of any length, so long as at least part of the molecule hybridizes sufficiently and specifically to GPR64 mRNA. The nucleic acid molecule can bind to any region of GPR64 mRNA. In some embodiments, the nucleotide sequence of GPR64 cDNA is shown in SEQ ID NO: 1 (NCBI Reference Sequence: NM_001079858.2). The amino acid sequence of Homo sapiens GPR64/ADGRG2 isoform 1 (longest isoform) is shown in SEQ ID NO:2.

In some embodiments, a region of the nucleic acid molecule is at least 70%, 75%, 80%, 85%, 90%, 91%, 92%, 93%, 94%, 95%, 96%, 97%, 98% or 99% complementary to at least a portion of SEQ ID NO:1. In some embodiments, the nucleic acid binds to at least a portion of nucleotides at positions 1815-1899, 2004-2064, 2067-2079, 2082-2142, 2277-2367, 2370-2430, 2505-2565, 2568-2571 or 2574-2634 of SEQ ID NO:1. In some embodiments, the composition can comprise a DNA molecule, such as an antisense DNA molecule. In some embodiments, the composition can comprise an RNA molecule, such as an anti-sense RNA molecule, a small interfering RNA (siRNA) molecule, or small hairpin RNA (shRNA) molecule, which may or may not be comprised on a vector, including a viral vector (such as an adeno-associated viral vector, an adenoviral vector, a retroviral vector, or a lentiviral vector) or a non-viral vector. In some embodiments, the expression of the DNA or RNA molecule may be regulated by a regulatory region specific to one or more types of cells present in the parathyroid gland.

A target sequence on a target mRNA can be selected from a given cDNA sequence corresponding to GPR64, in some embodiments, beginning 50 to 100 nt downstream (i.e., in the 3′ direction) from the start codon. In some embodiments, the target sequence can, however, be located in the 5′ or 3′ untranslated regions, or in the region nearby the start codon.

In one embodiment, the composition comprises a nucleic acid molecule that comprises a nucleotide sequence that binds to at least a portion of a nucleotide sequence of GPR64 mRNA. In some embodiments, the nucleic acid molecule is a DNA. In some embodiments, the nucleic acid molecule is an RNA.

In some embodiments, the composition comprises an anti-sense DNA. Anti-sense DNA binds with mRNA and prevents translation of the mRNA. The anti-sense DNA can be complementary to a portion of GPR64 mRNA. In some embodiments, the anti-sense DNA is complementary to the entire reading frame of GPR64. In some embodiments, the anti-sense DNA is complementary to the entire reading frame of SEQ ID NO:1. In some embodiments, the antisense DNA is complementary to a portion of SEQ ID NO:1. In some embodiments, the antisense DNA is at least 15 nucleotides, at least 20 nucleotides, at least 25 nucleotides, at least 30 nucleotides, at least 35 nucleotides, at least 40 nucleotides, at least 50 nucleotides, at least 75 nucleotides, at least 100 nucleotides, at least 150 nucleotides, at least 200 nucleotides, at least 300 nucleotides, at least 400 nucleotides, at least 500 nucleotides, at least 600 nucleotides, at least 700 nucleotides, at least 800 nucleotides, at least 900 nucleotides, at least 1000 nucleotides, at least 1500 nucleotides, at least 2000 nucleotides, at least 2500 nucleotides, at least 3000 nucleotides, at least 3500 nucleotides, at least 4000 nucleotides, or at least 4500 nucleotides.

In some embodiments, the composition comprises an anti-sense RNA. Anti-sense RNA binds with mRNA and prevents translation of the mRNA. The anti-sense RNA can be complementary to a portion of GPR64 mRNA. In some embodiments, the anti-sense RNA is complementary to the entire reading frame of GPR64. In some embodiments, the anti-sense RNA is complementary to the entire reading frame of SEQ ID NO:1. In some embodiments, the antisense RNA is complementary to a portion of SEQ ID NO:1. In some embodiments, the antisense RNA is at least 15 nucleotides, at least 20 nucleotides, at least 25 nucleotides, at least 30 nucleotides, at least 35 nucleotides, at least 40 nucleotides, at least 50 nucleotides, at least 75 nucleotides, at least 100 nucleotides, at least 150 nucleotides, at least 200 nucleotides, at least 300 nucleotides, at least 400 nucleotides, at least 500 nucleotides, at least 600 nucleotides, at least 700 nucleotides, at least 800 nucleotides, at least 900 nucleotides, at least 1000 nucleotides, at least 1500 nucleotides, at least 2000 nucleotides, at least 2500 nucleotides, at least 3000 nucleotides, at least 3500 nucleotides, at least 4000 nucleotides, or at least 4500 nucleotides.

In some embodiments, the composition is an siRNA targeting GPR64. SiRNAs are small single or dsRNAs that do not significantly induce the antiviral response common among vertebrate cells but that do induce target mRNA degradation via the RNAi pathway. The term siRNA refers to RNA molecules that have either at least one double stranded region or at least one single stranded region and possess the ability to effect RNA interference (RNAi). It is specifically contemplated that siRNA can refer to RNA molecules that have at least one double stranded region and possess the ability to effect RNAi. The dsRNAs (siRNAs) may be generated by various methods including chemical synthesis, enzymatic synthesis of multiple templates, digestion of long dsRNAs by a nuclease with RNAse III domains, and the like. An “siRNA directed to” at least a particular region of GPR64 means that a particular GPR64 siRNA includes sequences that result in the reduction or elimination of expression of the target gene, i.e., the siRNA is targeted to the region or gene.

The nucleotide sequence of the siRNA is defined by the nucleotide sequence of its target gene. The GPR64 siRNA contains a nucleotide sequence that is essentially identical to at least a portion of the target gene. In some embodiments, the siRNA contains a nucleotide sequence that is completely identical to at least a portion of the GPR64 gene. Of course, when comparing an RNA sequence to a DNA sequence, an “identical” RNA sequence will contain ribonucleotides where the DNA sequence contains deoxyribonucleotides, and further that the RNA sequence will typically contain a uracil at positions where the DNA sequence contains thymidine.

In some embodiments, a GPR64 siRNA comprises a double stranded structure, the sequence of which is “substantially identical” to at least a portion of the target gene. “Identity,” as known in the art, is the relationship between two or more polynucleotide (or polypeptide) sequences, as determined by comparing the sequences. In the art, identity also means the degree of sequence relatedness between polynucleotide sequences, as determined by the match of the order of nucleotides or amino acids between such sequences. Unless otherwise stated, sequence identity/similarity values provided herein refer to the value obtained using the BLAST 2.0 suite of programs using default parameters (Altschul, et al., (1997) Nucleic Acids Res. 25:3389-402).

One of skill in the art will appreciate that two polynucleotides of different lengths may be compared over the entire length of the longer fragment. Alternatively, small regions may be compared. Normally sequences of the same length are compared for a final estimation of their utility in the practice of the present invention. In some embodiments, there is 100% sequence identity between the dsRNA for use as siRNA and at least 15 contiguous nucleotides of the target gene, although a dsRNA having 70%, 75%, 80%, 85%, 90%, or 95% or greater may also be used in the present invention. A siRNA that is essentially identical to a least a portion of the target gene may also be a dsRNA wherein one of the two complementary strands (or, in the case of a self-complementary RNA, one of the two self-complementary portions) is either identical to the sequence of that portion or the target gene or contains one or more insertions, deletions or single point mutations relative to the nucleotide sequence of that portion of the target gene. siRNA technology thus has the property of being able to tolerate sequence variations that might be expected to result from genetic mutation, strain polymorphism, or evolutionary divergence.

In some embodiments, the invention provides an GPR64 siRNA that is capable of triggering RNA interference, a process by which a particular RNA sequence is destroyed (also referred to as gene silencing). In specific embodiments, GPR64 siRNA are dsRNA molecules that are 100 bases or fewer in length (or have 100 base pairs or fewer in its complementarity region). In some embodiments, a dsRNA may be 15, 16, 17, 18, 19, 20, 21, 22, 23, 24, 25, 26, 27, 28, 29, 30, 40, 50, 60, 70, 80, 90, 100 110, 120, 130, 140, 150, 160, 170, 180, 190, 200, 225, 250, 275, 300, 325, 350, 375, 400, 450, 500, 550, 600, 650, 700, 750, 800, 850, 900, 950, or 1000 nucleotides or more in length. In certain embodiments, GPR64 siRNA may be approximately 21 to 25 nucleotides in length. In some cases, it has a two nucleotide 3′ overhang and a 5′ phosphate. The particular GPR64 RNA sequence is targeted as a result of the complementarity between the dsRNA and the particular GPR64 RNA sequence. It will be understood that dsRNA or siRNA of the disclosure can effect at least a 20, 30, 40, 50, 60, 70, 80, 90 percent or more reduction of expression of a targeted GPR64 RNA in target cell. dsRNA of the invention (the term “dsRNA” will be understood to include “siRNA” and/or “candidate siRNA”) is distinct and distinguishable from antisense and ribozyme molecules by virtue of the ability to trigger RNAi. Structurally, dsRNA molecules for RNAi differ from antisense and ribozyme molecules in that dsRNA has at least one region of complementarity within the RNA molecule. In some embodiments, the complementary (also referred to as “complementarity”) region comprises at least or at most 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 contiguous bases. In some embodiments, long dsRNA are employed in which “long” refers to dsRNA that are 1000 bases or longer (or 1000 base pairs or longer in complementarity region). The term “dsRNA” includes “long dsRNA”, “intermediate dsRNA” or “small dsRNA” (lengths of 2 to 100 bases or base pairs in complementarity region) unless otherwise indicated. In some embodiments, dsRNA can exclude the use of siRNA, long dsRNA, and/or “intermediate” dsRNA (lengths of 100 to 1000 bases or base pairs in complementarity region).

It is specifically contemplated that a dsRNA may be a molecule comprising two separate RNA strands in which one strand has at least one region complementary to a region on the other strand. Alternatively, a dsRNA includes a molecule that is single stranded yet has at least one complementarity region as described above (such as when a single strand with a hairpin loop is used as a dsRNA for RNAi). For convenience, lengths of dsRNA may be referred to in terms of bases, which simply refers to the length of a single strand or in terms of base pairs, which refers to the length of the complementarity region. It is specifically contemplated that embodiments discussed herein with respect to a dsRNA comprised of two strands are contemplated for use with respect to a dsRNA comprising a single strand, and vice versa. In a two-stranded dsRNA molecule, the strand that has a sequence that is complementary to the targeted mRNA is referred to as the “antisense strand” and the strand with a sequence identical to the targeted mRNA is referred to as the “sense strand.” Similarly, with a dsRNA comprising only a single strand, it is contemplated that the “antisense region” has the sequence complementary to the targeted mRNA, while the “sense region” has the sequence identical to the targeted mRNA. Furthermore, it will be understood that sense and antisense region, like sense and antisense strands, are complementary (i.e., can specifically hybridize) to each other.

Strands or regions that are complementary may or may not be 100% complementary (“completely or fully complementary”). It is contemplated that sequences that are “complementary” include sequences that are at least 50% complementary, and may be at least 50%, 60%, 70%, 80%, or 90% complementary. In some embodiments, siRNA generated from sequence based on one organism may be used in a different organism to achieve RNAi of the cognate target gene. In other words, siRNA generated from a dsRNA that corresponds to a human gene may be used in a mouse cell if there is the requisite complementarity, as described above. Ultimately, the requisite threshold level of complementarity to achieve RNAi is dictated by functional capability. It is specifically contemplated that there may be mismatches in the complementary strands or regions. Mismatches may number at most or at least 1, 2, 3, 4, 5, 6, 7, 8, 9, 10, 11, 12, 13, 14, 15, 16, 17, 18, 19, 20, 21, 22, 23, 24, 25 residues or more, depending on the length of the complementarity region.

In some embodiments, the single RNA strand or each of two complementary double strands of a dsRNA molecule may be of at least or at most the following lengths: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, or more (including the full-length GPR64 mRNA without the poly-A tail) bases or base pairs. If the dsRNA is composed of two separate strands, the two strands may be the same length or different lengths. If the dsRNA is a single strand, in addition to the complementarity region, the strand may have 1, 2, 3, 4, 5, 6, 7, 8, 9, 10, 11, 12, 13, 14, 15, 16, 17, 18, 19, 20, 21, 22, 23, 24, 25, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more bases on either or both ends (5′ and/or 3′) or as forming a hairpin loop between the complementarity regions.

In some embodiments, the strand or strands of dsRNA are 100 bases (or base pairs) or less. In specific embodiments the strand or strands of the dsRNA are less than 70 bases in length. With respect to those embodiments, the dsRNA strand or strands may be from 5-70, 10-65, 20-60, 30-55, 40-50 bases or base pairs in length. A dsRNA that has a complementarity region equal to or less than 30 base pairs (such as a single stranded hairpin RNA in which the stem or complementary portion is less than or equal to 30 base pairs) or one in which the strands are 30 bases or fewer in length is specifically contemplated, as such molecules evade a mammalian's cell antiviral response. Thus, a hairpin dsRNA (one strand) may be 70 or fewer bases in length with a complementary region of 30 base pairs or fewer. In some cases, a dsRNA may be processed in the cell into siRNA.

The siRNA of the invention can comprise partially purified RNA, substantially pure RNA, synthetic RNA, or recombinantly produced RNA, as well as altered RNA that differs from naturally-occurring RNA by the addition, deletion, substitution and/or alteration of one or more nucleotides. Such alterations can include addition of non-nucleotide material, such as to the end(s) of the siRNA or to one or more internal nucleotides of the siRNA, including modifications that make the siRNA resistant to nuclease digestion.

One or both strands of the siRNA of the disclosure can comprise a 3′ overhang. As used herein, a “3′ overhang” refers to at least one unpaired nucleotide extending from the 3′-end of a duplexed RNA strand.

Thus in some embodiments, the GPR64 siRNA of the invention comprises at least one 3′ overhang of from 1 to about 6 nucleotides (which includes ribonucleotides or deoxynucleotides) in length, from 1 to about 5 nucleotides in length, from 1 to about 4 nucleotides in length, or from about 2 to about 4 nucleotides in length.

In some embodiments in which both strands of the GPR64 siRNA molecule comprise a 3′ overhang, the length of the overhangs can be the same or different for each strand. In some embodiments, the 3′ overhang is present on both strands of the siRNA, and is 2 nucleotides in length. For example, each strand of the GPR64 siRNA of the invention can comprise 3′ overhangs of dithymidylic acid (“TT”) or diuridylic acid (“UU”).

In order to enhance the stability of the present GPR64 siRNA, the 3′ overhangs can be also stabilized against degradation. In some embodiments, the overhangs are stabilized by including purine nucleotides, such as adenosine or guanosine nucleotides. In some embodiments, substitution of pyrimidine nucleotides by modified analogues, e.g., substitution of uridine nucleotides in the 3′ overhangs with 2′-deoxythymidine, is tolerated and does not affect the efficiency of RNAi degradation. In particular, the absence of a 2′ hydroxyl in the 2′-deoxythymidine can significantly enhance the nuclease resistance of the 3′ overhang in tissue culture medium.

In some embodiments, the GPR64 siRNA of the disclosure can be targeted to any stretch of approximately 19-25 contiguous nucleotides in any of the target mRNA sequences (the “target sequence”). Techniques for selecting target sequences for siRNA are given, for example, in Tuschl T et al., “The siRNA User Guide,” revised Oct. 11, 2002, the entire disclosure of which is herein incorporated by reference. “The siRNA User Guide” is available on the world wide web at a website maintained by Dr. Thomas Tuschl, Department of Cellular Biochemistry, AG 105, Max-Planck-Institute for Biophysical Chemistry, 37077 Gottingen, Germany, and can be found by accessing the website of the Max Planck Institute and searching with the keyword “siRNA.” Thus, in some embodiments, the sense strand of the present siRNA comprises a nucleotide sequence identical to any contiguous stretch of about 19 to about 25 nucleotides in the target mRNA.

In some embodiments of the invention, the GPR64 siRNA targets the GPR64 ORF sequence found within any of nucleotides 1815-1899, nucleotides 2004-2064, nucleotides 2067-2079, nucleotides 2082-2142, nucleotides 2277-2367, nucleotides 2370-2430, nucleotides 2505-2565, nucleotides 2568-2571 or nucleotides 2574-2634 of SEQ ID NO:1. In some embodiments, the siRNA comprises a 21 nucleotide double stranded sequence. In some embodiments, the siRNA comprises a two-TT overhang (Yang et al., Nucleic Acid Research, 34(4), 1224-1236, 2006).

In some embodiments, the composition useful in the methods of the invention comprises an shRNA molecule that targets GPR64 mRNA (GPR64 shRNA). shRNA is an artificial RNA molecule with a tight hairpin turn that can be used to silence target gene expression via RNA interference (RNAi). In certain cases, expression of GPR64 shRNA in cells is achieved through delivery of non-viral vectors (such as plasmids or bacterial vectors) or through viral vectors. shRNA is useful because it has a relatively low rate of degradation and turnover.

In order to obtain long-term gene silencing, expression vectors that continually express siRNAs in stably transfected mammalian cells can be used (Brummelkamp et al., Science 296: 550-553, 2002; Lee et al., Nature Biotechnol. 20:500-505, 2002; Miyagishi, M, and Taira, K. Nature Biotechnol. 20:497-500, 2002; Paddison, et al., Genes & Dev. 16:948-958, 2002; Paul et al., Nature Biotechnol. 20:505-508, 2002; Sui, Proc. Natl. Acad. Sci. USA 99(6):5515-5520, et al., 2002; Yu et al., Proc. Natl. Acad. Sci. USA 99(9):6047-6052, 2002). Many of these plasmids have been engineered to express shRNAs lacking poly (A) tails. Transcription of shRNAs is initiated at a polymerase III (pol III) promoter and is believed to be terminated at position 2 of a 4-5-thymine transcription termination site. Upon expression, shRNAs are thought to fold into a stem-loop structure with 3′ UU-overhangs. Subsequently, the ends of these shRNAs are processed, converting the shRNAs into ^{˜}21 nt siRNA-like molecules. The siRNA-like molecules can, in turn, bring about gene-specific silencing in the transfected mammalian cells.

The length of the stem and loop of shRNAs can be varied. In some embodiments, stem lengths could range anywhere from 25 to 29 nucleotides and loop size could range between 4 to 23 nucleotides without affecting silencing activity. Moreover, presence of G-U mismatches between the two strands of the shRNA stem does not necessarily lead to a decrease in potency.

In some embodiments, the present invention is directed to methods of administering subjects with compositions comprising expression vectors and/or chemically synthesized shRNA molecules that target GPR64. In some embodiments of the invention, the GPR64 shRNA targets the GPR64 ORF sequence found within any of nucleotides 1815-1899, nucleotides 2004-2064, nucleotides 2067-2079, nucleotides 2082-2142, nucleotides 2277-2367, nucleotides 2370-2430, nucleotides 2505-2565, nucleotides 2568-2571 or nucleotides 2574-2634 of SEQ ID NO:1. In some embodiments, the composition comprises a nucleotide sequence expressing a small hairpin RNA (shRNA) molecule. In some embodiments, the expression vector is a lentivirus expression vector.

In some embodiments, it is contemplated that nucleic acids or antibodies of the invention may be labeled. The label may be fluorescent, radioactive, enzymatic, or calorimetric. It is contemplated that a dsRNA may have one label attached to it or it may have more than one label attached to it. When more than one label is attached to a dsRNA, the labels may be the same or be different. If the labels are different, they may appear as different colors when visualized. The label may be on at least one end and/or it may be internal. Furthermore, there may be a label on each end of a single stranded molecule or on each end of a dsRNA made of two separate strands. The end may be the 3′ and/or the 5′ end of the nucleic acid. A label may be on the sense strand or the sense end of a single strand (end that is closer to sense region as opposed to antisense region), or it may be on the antisense strand or antisense end of a single strand (end that is closer to antisense region as opposed to sense region). In some cases, a strand is labeled on a particular nucleotide (G, A, U, or C). When two or more differentially colored labels are employed, fluorescent resonance energy transfer (FRET) techniques may be employed to characterize the dsRNA.

Labels contemplated for use in several embodiments are non-radioactive. In many embodiments of the invention, the labels are fluorescent, though they may be enzymatic, radioactive, or positron emitters. Fluorescent labels that may be used include, but are not limited to, BODIPY, Alexa Fluor, fluorescein, Oregon Green, tetramethylrhodamine, Texas Red, rhodamine, cyanine dye, or derivatives thereof. The labels may also more specifically be Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5, DAPI, 6-FAM, Killer Red, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, TET, Tetramethylrhodamine, and/or Texas Red. A labeling reagent is a composition that comprises a label and that can be incubated with the nucleic acid to effect labeling of the nucleic acid under appropriate conditions. In some embodiments, the labeling reagent comprises an alkylating agent and a dye, such as a fluorescent dye. In some embodiments, a labeling reagent comprises an alkylating agent and a fluorescent dye such as Cy3, Cy5, or fluorescein (FAM). In still further embodiments, the labeling reagent is also incubated with a labeling buffer, which may be any buffer compatible with physiological function (i.e., buffers that is not toxic or harmful to a cell or cell component) (termed “physiological buffer”).

In some embodiments, the nucleic acids of the invention can be modified. In some embodiments, the nucleic acids can be modified to include a phosphorothioate (PS) backbone. The modification to the backbone can be throughout the molecule or at one or more defined sites. In some embodiments, the nucleic acids can be modified to encompass peptide nucleic acids (PNA). In some embodiments, the nucleic acids can be modified to encompass phosphorodiamidate morpholino oligomers (PMO).

In some embodiments, the nucleic acid molecules of the invention can include derivatives such as S-oligonucleotides (phosphorothioate derivatives or S-oligos). S-oligos (nucleoside phosphorothioates) are isoelectronic analogs of an oligonucleotide (O-oligo) in which a nonbridging oxygen atom of the phosphate group is replaced by a sulfur atom. The S-oligos of the present invention may be prepared by treatment of the corresponding O-oligos with 3H-1,2-benzodithiol-3-one-1,1-dioxide which is a sulfur transfer reagent. See Iyer et al., J. Org. Chem. 55:4693-4698 (1990); and Iyer et al., J. Am. Chem. Soc. 112:1253-1254 (1990), the disclosures of which are fully incorporated by reference herein.

In some embodiments of the invention, a dsRNA has one or more non-natural nucleotides, such as a modified residue or a derivative or analog of a natural nucleotide. Any modified residue, derivative or analog may be used to the extent that it does not eliminate or substantially reduce (by at least 50%) RNAi activity of the dsRNA.

A person of ordinary skill in the art is well aware of achieving hybridization of complementary regions or molecules. Such methods typically involve heat and slow cooling of temperature during incubation, for example.

In some embodiments, the nucleic acid molecules of the present methods are encoded by expression vectors. The expression vectors may be obtained and introduced into a cell. Once introduced into the cell the expression vector is transcribed to produce various nucleic acids. Expression vectors include nucleic acids that provide for the transcription of a particular nucleic acid. Expression vectors include plasmid DNA, linear expression elements, circular expression elements, viral expression constructs (including adenoviral, adeno-associated viral, retroviral, lentiviral, and so forth), and the like, all of which are contemplated as being used in the compositions and methods of the present disclosure. In some embodiments one or at least 2, 3, 4, 5, 6, 7, 8, 9, 10 or more nucleic acid molecules binding to GPR64 RNA are encoded by a single expression construct. Expression of the nucleic acid molecules binding to GPR64 RNA may be independently controlled by at least 1, 2, 3, 4, 5, 6, 7, 8, 9, 10 or more regulatory elements. In certain embodiments, at least 1, 2, 3, 4, 5, 6, 7, 8, 9, 10 or more expression constructs can be introduced into a cell. Each expression construct can encode 1, 2, 3, 4, 5, 6, 7, 8, 9, 10 or more nucleic acid molecules binding to GPR64 RNA. In some embodiments, nucleic acid molecules binding to GPR64 RNA may be encoded as expression domains. Expression domains include a transcription control element, which may or may not be independent of other control or promoter elements; a nucleic acid; and optionally a transcriptional termination element.

In some embodiments, the invention provides a viral vector encoding GPR64 or a biologically active fragment or derivative thereof. In some embodiments, the viral vector comprises a nucleic acid sequence encoding GPR64 or a biologically active fragment or derivative thereof as provided herein. In some embodiments, the GPR64 or a biologically active fragment or derivative thereof encodes a protein that is at least 90% identical to SEQ ID NO:2. In some embodiments, the GPR64 or a biologically active fragment or derivative thereof may be derived from genomic DNA, i.e., cloned directly from the genome of a particular organism. In some embodiments, however, the vector comprising GPR64 comprises complementary DNA (cDNA).

The organismal source of GPR64 is not limiting. In some embodiments, the GPR64 nucleic acid sequence is derived from a mammal, bird, reptile or fish. In some embodiments, the GPR64 is of human origin. In some embodiments, the GPR64 is from dog, cat, horse, mouse, rat, guinea pig, sheep, cow, pig, monkey, or ape. The nucleic acid molecules may be produced using recombinant DNA technology (e.g., polymerase chain reaction (PCR) amplification, cloning) or chemical synthesis. GPR64 nucleic acids include natural nucleic acid molecules and homologues thereof, including, but not limited to, natural allelic variants and modified nucleic acid molecules in which nucleotides have been inserted, deleted, substituted, and/or inverted in such a manner that such modifications provide the desired effect. In some embodiments, the coding sequence of GPR64 is encoded by SEQ ID NO:1. “GPR64” nucleic acid in accordance with the invention may contain a variety of different bases compared to the wild-type sequence and yet still encode a corresponding polypeptide that exhibits the biological activity of the native GPR64 polypeptide.

In some embodiments, the vector comprises a nucleic acid sequence that is at least 60%, 70%, 75%, 80%, 85%, 90%, 91%, 92%, 93%, 94%, 95%, 96%, 97%, 98%, 99%, or 100% identical to the coding sequence of SEQ ID NO: 1. In some embodiments, the vector comprises a nucleic acid sequence that encodes a protein that is at least 80%, 85%, 90%, 91%, 92%, 93%, 94%, 95%, 96%, 97%, 98%, 99%, or 100% identical to the amino acid sequence of SEQ ID NO:2.

Any suitable viral vector can be used in the methods of the invention. For example, vectors derived from adenovirus (AV); adeno-associated virus (AAV; including AAV serotypes); retroviruses (e.g, lentiviruses (LV), Rhabdoviruses, murine leukemia virus); herpes virus, and the like. The tropism of the viral vectors can also be modified by pseudotyping the vectors with envelope proteins or other surface antigens from other viruses. For example, an AAV vector of the invention can be pseudotyped with surface proteins from vesicular stomatitis virus (VSV), rabies, Ebola, Mokola, and the like.

Selection of recombinant viral vectors suitable for use in the invention, are within the skill in the art. See, for example, Dornburg R (1995), Gene Therap. 2: 301-310; Eglitis M A (1988), Biotechniques 6: 608-614; Miller A D (1990), Hum Gene Therap. 1: 5-14; and Anderson W F (1998), Nature 392: 25-30, the entire disclosures of which are herein incorporated by reference.

The ability of a RNA of the claimed invention to cause RNAi-mediated degradation of the target mRNA can be evaluated using standard techniques for measuring the levels of RNA or protein in cells. For example, GPR64 siRNA of the invention can be delivered to cultured cells, and the levels of target mRNA can be measured by Northern blot or dot blotting techniques, or by quantitative RT-PCR. Alternatively, the levels of GPR64 protein in the cultured cells can be measured by ELISA or Western blot. A suitable cell culture system for measuring the effect of the present siRNA on target mRNA or protein levels may be utilized. RNAi-mediated degradation of GPR64 mRNA by an siRNA containing a given target sequence can also be evaluated with animal models, for example.

In other embodiments, the method comprises administering a composition comprising a polypeptide or antibody that reduces the activity of GPR64. As used herein, the term “antibody” includes any immunologic binding agent, such as IgG, IgM, IgA, IgD and IgE. The term “antibody” may be used to refer to any antibody-like molecule that has an antigen binding region, and includes antibody fragments such as Fab′, Fab, F(ab′)2, single domain antibodies (DABs), Fv, scFv (single chain Fv), and the like. Monoclonal and humanized antibodies are also contemplated in the disclosure.

In some embodiments, the nucleic acids can be administered to the subject either as naked nucleic acid, in conjunction with a delivery reagent, or as a recombinant plasmid or viral vector that expresses the nucleic acids. Delivery of nucleic acids or vectors to an individual may occur by any suitable means, but in specific embodiments it occurs by one of the following: cyclodextrin delivery system; ionizable lipids; DPC conjugates; GalNAc-conjugates; self-assembly of oligonucleotide nanoparticles (DNA tetrahedra carrying multiple siRNAs); or polymeric nanoparticles made of low-molecular-weight polyamines and lipids (see Kanasty et al. Nature Materials 12, 967-977 (2013) for review of same).

Suitable delivery reagents for administration in conjunction with the present nucleic acids or vectors include at least the Minis Transit TKO lipophilic reagent; lipofectin; lipofectamine; cellfectin; or polycations (e.g., polylysine), or liposomes. In specific embodiments, a particular delivery reagent comprises a liposome.

Liposomes can aid in the delivery of the present nucleic acids or vectors to a particular tissue, and can also increase the blood half-life of the nucleic acids. Liposomes suitable for use in the invention can be formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, for example as described in Szoka et al. (1980), Ann. Rev. Biophys. Bioeng. 9: 467; and U.S. Pat. Nos. 4,235,871, 4,501,728, 4,837,028, and 5,019,369, the entire disclosures of which are herein incorporated by reference.

In certain aspects, the liposomes encapsulating the present nucleic acids comprise a ligand molecule that can target the liposome to a particular cell or tissue at or near the site of interest. Ligands that bind to receptors prevalent in the tissues to be targeted, such as monoclonal antibodies that bind to surface antigens, are contemplated. In particular cases, the liposomes are modified so as to avoid clearance by the mononuclear macrophage and reticuloendothelial systems, for example by having opsonization-inhibition moieties bound to the surface of the structure. In one embodiment, a liposome of the invention can comprise both opsonization-inhibition moieties and a ligand. Opsonization-inhibiting moieties for use in preparing the liposomes of the disclosure are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer which significantly decreases the uptake of the liposomes by the macrophage-monocyte system (“MMS”) and reticuloendothelial system (“RES”); e.g., as described in U.S. Pat. No. 4,920,016, the entire disclosure of which is herein incorporated by reference. Liposomes modified with opsonization-inhibition moieties thus remain in the circulation much longer than unmodified liposomes. For this reason, such liposomes are sometimes called “stealth” liposomes.

Stealth liposomes are known to accumulate in tissues fed by porous or “leaky” microvasculature. Thus, target tissue characterized by such microvasculature defects, for example solid tumors, will efficiently accumulate these liposomes; see Gabizon, et al. (1988), P.N.A. S., USA, 18: 6949-53. In addition, the reduced uptake by the RES lowers the toxicity of stealth liposomes by preventing significant accumulation in the liver and spleen. Thus, liposomes of the invention that are modified with opsonization-inhibition moieties can deliver the present nucleic acids to tumor cells.

In some embodiments, opsonization inhibiting moieties suitable for modifying liposomes are water-soluble polymers with a number-average molecular weight from about 500 to about 40,000 Daltons, and in some embodiments from about 2,000 to about 20,000 Daltons. Such polymers can include polyethylene glycol (PEG) or polypropylene glycol (PPG) derivatives; e.g., methoxy PEG or PPG, and PEG or PPG stearate; synthetic polymers such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM1. Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups.

In some embodiments the opsonization-inhibiting moiety is a PEG, PPG, or derivatives thereof. Liposomes modified with PEG or PEG-derivatives are sometimes called “PEGylated liposomes.” The opsonization inhibiting moiety can be bound to the liposome membrane by any one of numerous well-known techniques. For example, an N-hydroxysuccinimide ester of PEG can be bound to a phosphatidyl-ethanolamine lipid-soluble anchor, and then bound to a membrane. Similarly, a dextran polymer can be derivatized with a stearylamine lipid-soluble anchor via reductive amination using Na(CN)BH₃ and a solvent mixture such as tetrahydrofuran and water in a 30:12 ratio at 60 degrees C.

Recombinant plasmids that express nucleic acids of the invention are discussed above. Such recombinant plasmids can also be administered directly or in conjunction with a suitable delivery reagent, including the Mirus Transit LT 1 lipophilic reagent; lipofectin; lipofectamine; cellfectin; polycations (e.g., polylysine) or liposomes.

The nucleic acids reducing the level of GPR64 of the invention can be administered to the subject by any suitable means. For example, the nucleic acids can be administered by gene gun, electroporation, or by other suitable parenteral or enteral administration routes, or by injection, for example, by intramuscular or intravenous injection.

Suitable parenteral administration routes include intravascular administration (e.g. intravenous bolus injection, intravenous infusion, intra-arterial bolus injection, intra-arterial infusion and catheter instillation into the vasculature); peri- and intra-tissue administration (e.g., peri-tumoral and intra-tumoral injection, intra-retinal injection or subretinal injection); subcutaneous injection or deposition including subcutaneous infusion (such as by osmotic pumps); direct (e.g., topical) application to the area at or near the site of interest, for example by a catheter or other placement device (e.g., a corneal pellet or a suppository, eye-dropper, or an implant comprising a porous, non-porous, or gelatinous material); and inhalation. In a particular embodiment, injections or infusions of the composition(s) are given at or near the site of disease.

The nucleic acids reducing the level of GPR64 of the invention can be administered in a single dose or in multiple doses. Where the administration of a composition is by infusion, the infusion can be a single sustained dose or can be delivered by multiple infusions. Injection of the agent directly into the tissue is at or near the site of need. Multiple injections of the agent into the tissue at or near the site of interest are encompassed within this disclosure.

One skilled in the art can also readily determine an appropriate dosage regimen for administering the nucleic acids reducing the level of GPR64 of the invention to a given subject. For example, the composition(s) can be administered to the subject once, such as by a single injection or deposition at or near the site of interest. In some embodiments, the composition(s) can be administered to a subject once or twice daily to a subject once weekly for a period of from about three to about twenty-eight days, in some embodiments, from about seven to about ten weeks. In some dosage regimens, the composition(s) is injected at or near the site of interest once a day for seven days. Where a dosage regimen comprises multiple administrations, it is understood that the effective amount of composition(s) administered to the subject can comprise the total amount of composition(s) administered over the entire dosage regimen.

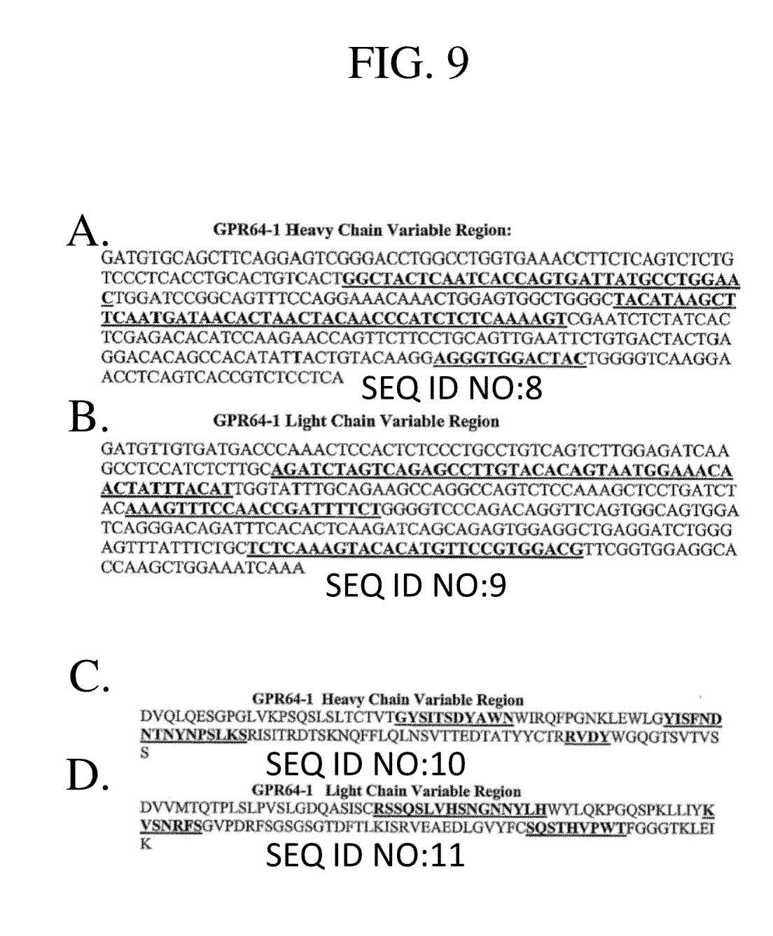

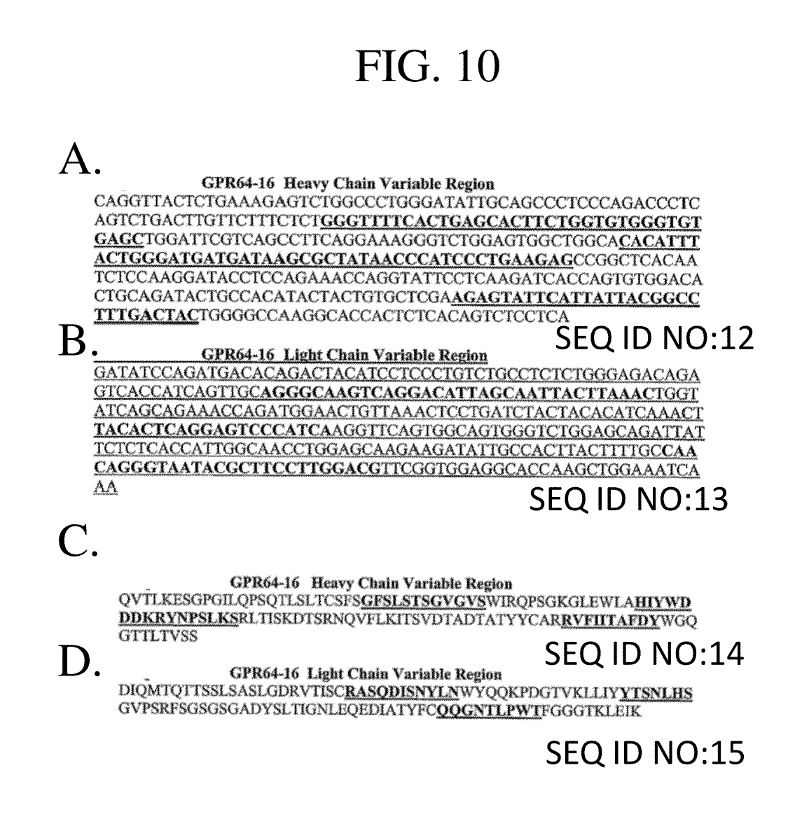

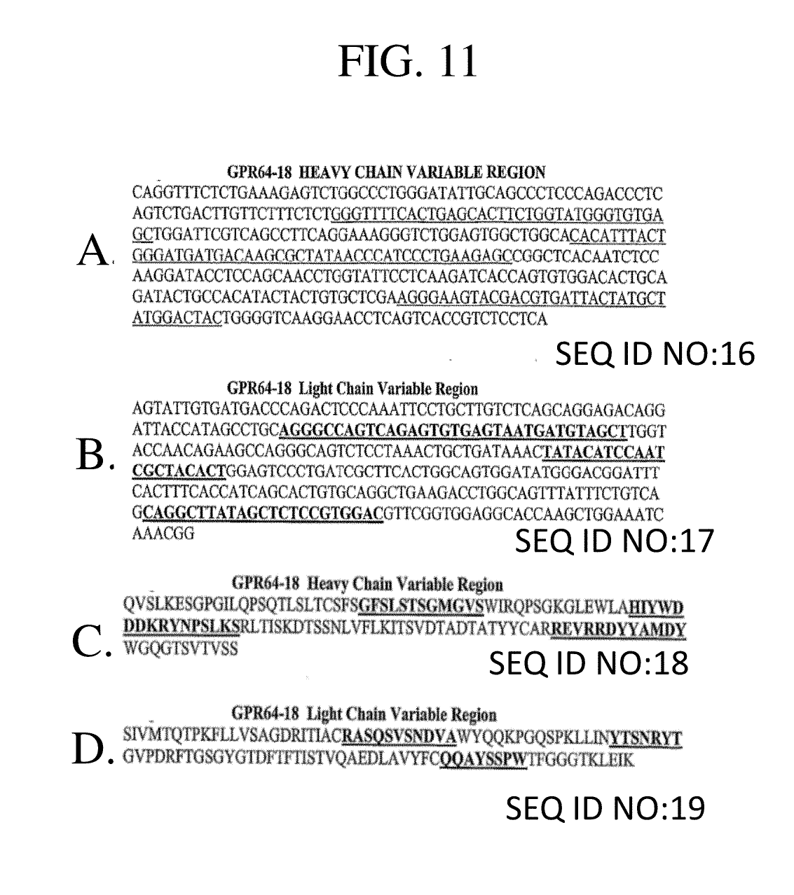

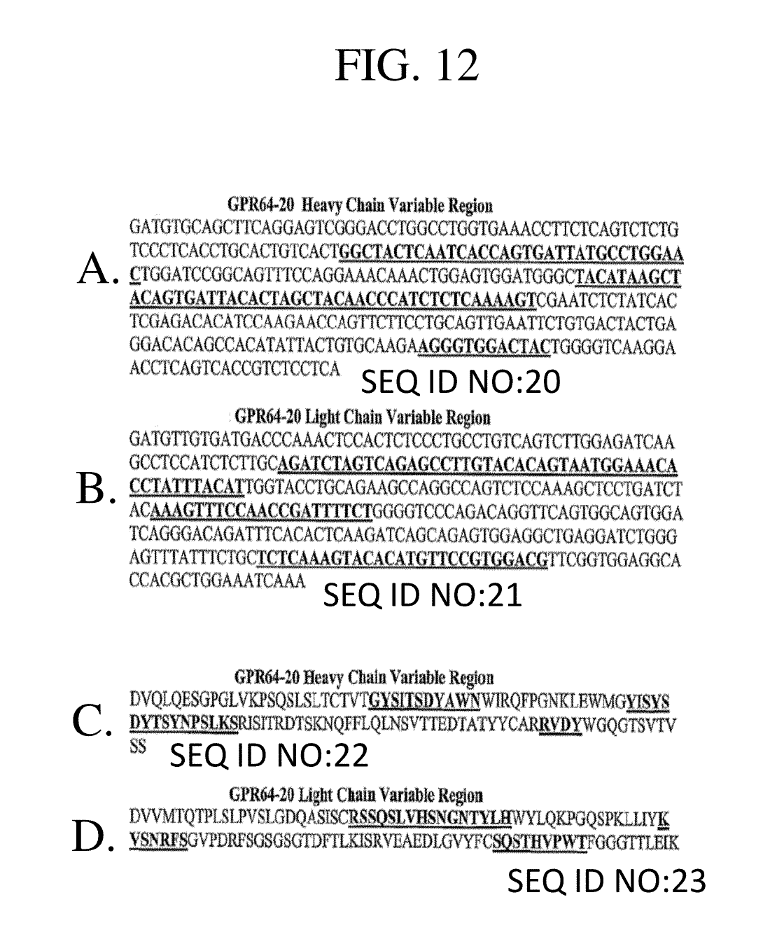

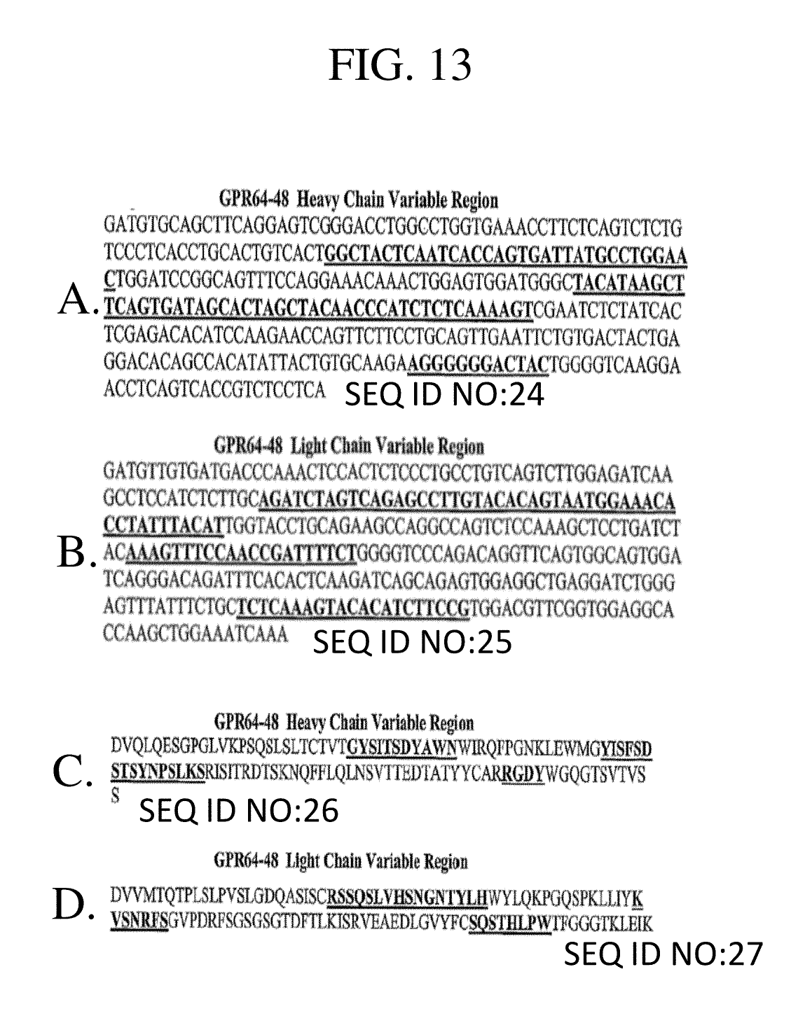

In some embodiments, the composition useful in some methods of the invention comprises an antibody or biologically active fragment thereof that binds to GPR64 and inhibits its activity. In some embodiments, the antibody can include any of the GPR64 antibodies described in WO 2004/058171, which is incorporated by reference herein. The antibody variable region nucleotide and amino acid sequences, including the complementarity determining regions (CDR) are shown in FIGS. 9-13. In one embodiment, the antibody comprises a heavy chain variable region comprising any of SEQ ID NOS: 10, 14, 18, 22, or 26. In one embodiment, the antibody comprises the CDRs from a heavy chain variable region comprising any of SEQ ID NOS: 10, 14, 18, 22, or 26. In one embodiment, the antibody comprises a light chain variable region comprising any of SEQ ID NOS: 11, 15, 19, 23, or 27. In one embodiment, the antibody comprises the CDRs from a light chain variable region comprising any of SEQ ID NOS: 11, 15, 19, 23, or 27.

### II. Pharmaceutical Compositions

Where clinical applications are contemplated, it will be necessary to prepare pharmaceutical compositions in a form appropriate for the intended application. Generally, this will entail preparing compositions that are suitable for administration to a subject, e.g., essentially free of pyrogens, as well as other impurities that could be harmful to humans or animals.

In one embodiment, the present invention provides a composition for treating hypercalcemia comprising an agent that decreases the level and/or activity of GPR64 and a pharmaceutically acceptable carrier.

In one embodiment, the present invention provides a composition for treating hypercalcemia comprising a nucleic acid that decreases the level and/or activity of GPR64 and a pharmaceutically acceptable carrier.

In some embodiments, the invention provides a pharmaceutical composition comprising a viral vector encoding a nucleic acid that decreases the level and/or activity of GPR64 and a pharmaceutically acceptable carrier.

In some embodiments, the invention provides a pharmaceutical composition comprising an antibody that decreases the activity of GPR64 and a pharmaceutically acceptable carrier.

In some embodiments, the invention provides a pharmaceutical composition comprising a therapeutically effective amount of an agonist or activator of GPR64 and a pharmaceutically acceptable carrier. In some embodiments, the agonist or activator is a peptide (P-15) comprising the sequence TSFGVLLDLSRTSVL (SEQ ID NO:3), or a biologically active fragment or derivative thereof.

In some embodiments, the invention provides a pharmaceutical composition comprising a therapeutically effective amount of a vector encoding GPR64 or a biologically active fragment or derivative thereof and a pharmaceutically acceptable carrier.

In some embodiments, the composition comprises appropriate salts and/or buffers to render delivery of nucleic acid, vectors or antibodies stable and allow for binding to or uptake by target cells. In some embodiments, the compositions are dispersed in a pharmaceutically acceptable carrier or aqueous medium. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. Except insofar as any conventional media or agent is incompatible with the agents of the present technology, its use in therapeutic compositions is contemplated. Supplementary active ingredients also can be incorporated into the compositions.

The active compositions of the present technology may include classic pharmaceutical preparations. Administration of these compositions according to the present technology will be via any common route so long as the target tissue is available via that route. Such routes of administration may include oral, parenteral (including intravenous, intramuscular, subcutaneous, intradermal, intra-articular, intra-synovial, intrathecal, intra-arterial, intracardiac, subcutaneous, intraorbital, intracapsular, intraspinal, intrastemal, and transdermal), nasal, buccal, urethral, rectal, vaginal, mucosal, dermal, or topical (including dermal, buccal, and sublingual). Alternatively, administration may be by orthotopic, intradermal, subcutaneous, intramuscular, intraperitoneal or intravenous injection. Such compositions would normally be administered as pharmaceutically acceptable compositions. Of particular interest is direct administration to parathyroid glands, perfusion of the gland, or a local or regional administration, for example, in the local or regional vasculature or lymphatic system. Administration can also be via nasal spray, surgical implant, internal surgical paint, infusion pump, or via catheter, stent, balloon or other delivery device. The most useful and/or beneficial mode of administration can vary, especially depending upon the condition of the recipient and the disorder being treated.

In some embodiments, compositions which are dispersions can also be prepared, e.g., in glycerol, liquid polyethylene glycols, and mixtures thereof and in oils. Under ordinary conditions of storage and use, these preparations may contain a preservative to prevent the growth of microorganisms.

In some embodiments, pharmaceutical forms suitable for injectable use include sterile aqueous solutions or dispersions and sterile powders for the extemporaneous preparation of sterile injectable solutions or dispersions. In all cases, the form must be sterile and should be fluid to the extent that easy syringability exists. In some embodiments, it must be stable under the conditions of manufacture and storage and must be preserved against the contaminating action of microorganisms, such as bacteria and fungi. In some embodiments, the carrier can be a solvent or dispersion medium containing, for example, water, ethanol, polyol (e.g., glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In some embodiments, it will be preferable to include isotonic agents, for example, sugars or sodium chloride. Prolonged absorption of the injectable compositions can be brought about by the use in the compositions of agents delaying absorption, for example, aluminum monostearate and gelatin.

In some embodiments, sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions can be administered in a variety of dosage forms. Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by FDA Office of Biologics standards.

For oral administration the agents may be incorporated with excipients and used in the form of non-ingestible mouthwashes and dentifrices. It is anticipated that virtually any pill or capsule type known to one of skill in the art including, e.g., coated, and time delay, slow release, etc., may be used with the present technology. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, creams, powders and slurries. The active ingredient may be added in a therapeutically effective amount to a paste dentifrice that may include water, binders, abrasives, flavoring agents, foaming agents, and humectants.

Pharmaceutical compositions suitable for oral dosage may take various forms, such as tablets, capsules, caplets, and wafers (including rapidly dissolving or effervescing), each containing a predetermined amount of the active agent. The compositions may also be in the form of a powder or granules, a solution or suspension in an aqueous or non-aqueous liquid, and as a liquid emulsion (oil-in-water and water-in-oil). The active agents may also be delivered as a bolus, electuary, or paste. It is generally understood that methods of preparations of the above dosage forms are generally known in the art, and any such method would be suitable for the preparation of the respective dosage forms for use in delivery of the compositions.

Hard capsules containing the compositions may be made using a physiologically degradable composition, such as gelatin. Such hard capsules comprise the compound, and may further comprise additional ingredients including, for example, an inert solid diluent such as calcium carbonate, calcium phosphate, or kaolin. Soft gelatin capsules containing the compound may be made using a physiologically degradable composition, such as gelatin. Such soft capsules comprise the compound, which may be mixed with water or an oil medium such as peanut oil, liquid paraffin, or olive oil.

Sublingual tablets are designed to dissolve very rapidly. Examples of such compositions include ergotamine tartrate, isosorbide dinitrate, and isoproterenol HCL. The compositions of these tablets contain, in addition to the agent, various soluble excipients, such as lactose, powdered sucrose, dextrose, and mannitol. The solid dosage forms of the present technology may optionally be coated, and examples of suitable coating materials include, but are not limited to, cellulose polymers (such as cellulose acetate phthalate, hydroxypropyl cellulose, hydroxypropyl methylcellulose, hydroxypropyl methylcellulose phthalate, and hydroxypropyl methylcellulose acetate succinate), polyvinyl acetate phthalate, acrylic acid polymers and copolymers, and methacrylic resins (such as those commercially available under the trade name EUDRAGIT), zein, shellac, and polysaccharides.

Powdered and granular compositions of a pharmaceutical preparation may be prepared using known methods. Such compositions may be administered directly to a patient or used in the preparation of further dosage forms, such as to form tablets, fill capsules, or prepare an aqueous or oily suspension or solution by addition of an aqueous or oily vehicle thereto. Each of these compositions may further comprise one or more additives, such as dispersing or wetting agents, suspending agents, and preservatives. Additional excipients (e.g., fillers, sweeteners, flavoring, or coloring agents) may also be included in these compositions.

Liquid compositions of pharmaceutical compositions which are suitable for oral administration may be prepared, packaged, and sold either in liquid form or in the form of a dry product intended for reconstitution with water or another suitable vehicle prior to use.

A tablet containing one or more active agent compounds described herein may be manufactured by any standard process readily known to one of skill in the art, such as, for example, by compression or molding, optionally with one or more adjuvant or accessory ingredient. The tablets may optionally be coated or scored and may be formulated so as to provide slow or controlled release of the active agents.

Solid dosage forms may be formulated so as to provide a delayed release of the active agents, such as by application of a coating. Delayed release coatings are known in the art, and dosage forms containing such may be prepared by any known suitable method. Such methods generally include that, after preparation of the solid dosage form (e.g., a tablet or caplet), a delayed release coating composition is applied. Application can be by methods, such as airless spraying, fluidized bed coating, use of a coating pan, or the like. Materials for use as a delayed release coating can be polymeric in nature, such as cellulosic material (e.g., cellulose butyrate phthalate, hydroxypropyl methylcellulose phthalate, and carboxymethyl ethylcellulose), and polymers and copolymers of acrylic acid, methacrylic acid, and esters thereof.

Solid dosage forms according to the present technology may also be sustained release (i.e., releasing the active agents over a prolonged period of time), and may or may not also be delayed release. Sustained release compositions are known in the art and are generally prepared by dispersing a drug within a matrix of a gradually degradable or hydrolyzable material, such as an insoluble plastic, a hydrophilic polymer, or a fatty compound. Alternatively, a solid dosage form may be coated with such a material.

Compositions for parenteral administration include aqueous and non-aqueous sterile injection solutions, which may further contain additional agents, such as antioxidants, buffers, bacteriostats, and solutes, which render the compositions isotonic with the blood of the intended recipient. The compositions may include aqueous and non-aqueous sterile suspensions, which contain suspending agents and thickening agents. Such compositions for parenteral administration may be presented in unit-dose or multi-dose containers, such as, for example, sealed ampoules and vials, and may be stores in a freeze-dried (lyophilized) condition requiring only the addition of the sterile liquid carrier, for example, water (for injection), immediately prior to use. Extemporaneous injection solutions and suspensions may be prepared from sterile powders, granules, and tablets of the kind previously described.

Compositions for rectal delivery include rectal suppositories, creams, ointments, and liquids. Suppositories may be presented as the active agents in combination with a carrier generally known in the art, such as polyethylene glycol. Such dosage forms may be designed to disintegrate rapidly or over an extended period of time, and the time to complete disintegration can range from a short time, such as about 10 minutes, to an extended period of time, such as about 6 hours.

Topical compositions may be in any form suitable and readily known in the art for delivery of active agents to the body surface, including dermally, buccally, and sublingually. Typical examples of topical compositions include ointments, creams, gels, pastes, and solutions. Compositions for administration in the mouth include lozenges.

In accordance with these embodiments, oral (topical, mucosal, and/or dermal) delivery materials can also include creams, salves, ointments, patches, liposomes, nanoparticles, microparticles, timed-release formulations and other materials known in the art for delivery to the oral cavity, mucosa, and/or to the skin of a subject for treatment and/or prevention of a condition disclosed herein. Certain embodiments concern the use of a biodegradable oral (topical, mucosal, and/or dermal) patch delivery system or gelatinous material. These compositions can be a liquid formulation or a pharmaceutically acceptable delivery system treated with a formulation of these compositions, and may also include activator/inducers.

The compositions for use in the methods of the present technology may also be administered transdermally, wherein the active agents are incorporated into a laminated structure (generally referred to as a “patch”) that is adapted to remain in intimate contact with the epidermis of the recipient for a prolonged period of time. Typically, such patches are available as single layer “drug-in-adhesive” patches or as multi-layer patches where the active agents are contained in a layer separate from the adhesive layer. Both types of patches also generally contain a backing layer and a liner that is removed prior to attachment to the recipient's skin. Transdermal drug delivery patches may also be comprised of a reservoir underlying the backing layer that is separated from the skin of the recipient by a semi-permeable membrane and adhesive layer. Transdermal drug delivery may occur through passive diffusion, electrotransport, or iontophoresis.

In certain embodiments, a patch contemplated herein may be a slowly dissolving or a time-released patch. In accordance with these embodiments, a slowly dissolving patch can be an alginate patch. In certain examples, a patch may contain a detectible indicator dye or agent such as a fluorescent agent. In other embodiments, a tag (e.g., detectible tag such as a biotin or fluorescently tagged agent) can be associated with a treatment molecule in order to detect the molecule after delivery to the subject. In certain embodiments, one or more oral delivery patches or other treatment contemplated herein may be administered to a subject three times daily, twice daily, once a day, every other day, weekly, and the like, depending on the need of the subject as assessed by a health professional. Patches contemplated herein may be oral-biodegradable patches or patches for exterior use that may or may not degrade. Patches contemplated herein may be 1 mm, 2 mm, 3 mm, 4 mm to 5 mm in size or more depending on need.

In some embodiments, compositions may include short-term, rapid-onset, rapid-offset, controlled release, sustained release, delayed release, and pulsatile release compositions, providing the compositions achieve administration of the agents as described herein. See Remington's Pharmaceutical Sciences (18th ed.; Mack Publishing Company, Eaton, Pa., 1990), herein incorporated by reference in its entirety.

In certain embodiments, the compositions disclosed herein can be delivered via a medical device. Such delivery can generally be via any insertable or implantable medical device, including, but not limited to stents, catheters, balloon catheters, shunts, or coils. In one embodiment, the present technology provides medical devices, such as stents, the surface of which is coated with a compound or composition as described herein. The medical device of this technology can be used, for example, in any application for treating, preventing, or otherwise affecting the course of a disease or condition, such as those disclosed herein.

### III. Screening Assays

In some embodiments, the invention provides methods of screening for inhibitors or agonists of GPR64.

By “agonist” is intended naturally occurring and/or synthetic compounds capable of increasing the expression level or activating GPR64. In some embodiments, the agonist is capable of antagonizing CaSR-mediated inactivation of adenylate cyclase, stimulating PTH release mediated by GPR64, enhancing production of cAMP, increasing extracellular calcium concentrations, or other biological activities of the protein.

By “inhibitor” is intended naturally occurring and/or synthetic agents capable of reducing the expression level or activity of GPR64. In some embodiments, the inhibitor is capable of decreasing production of cAMP, inhibiting parathyroid hormone secretion, decreasing extracellular calcium concentrations, or other biological activity of the protein.

In some embodiments, an assay for GPR64 activity in cells can be used to determine the functionality of GPR64 in the presence of an agent which may act as an inhibitor or agonist, and thus, agents that interfere with or enhance the expression level or activity of GPR64 can be identified.

Assays performed in animals, such as mice, are also included as part of the screening methods provided herein. In some embodiments, transgenic animals expressing wild-type receptor or expressing a mutant receptor in parathyroid glands is used for screening agonists and/or antagonists, followed by measuring circulating PTH.

In some embodiments, GPR64 is employed in a screening process for compounds which bind the protein and which enhances (agonists) or inhibits (antagonists) the activity of GPR64. Thus, in some embodiments, GPR64 is used to assess the binding of molecular substrates and ligands in, for example, cells, cell-free preparations, chemical libraries, and natural product mixtures. These substrates and ligands can be natural substrates and ligands or can be structural or functional mimetics. Inhibitors of GPR64 are particularly advantageous and can be used in methods as therapeutic agents in the treatment of diseases or conditions, such as hypercalcemia, as described herein.

In some embodiments, the screening procedures involve producing appropriate cells which express GPR64. Such cells can include cells from mammals, yeast, Drosophila or E. coli. In some embodiments, the cells express the polypeptide endogenously. In other embodiments, the cells have been transfected or engineered to express the polypeptide. In some embodiments, the cells are parathyroid glandular cells. In some embodiments, the cells are human embryonic kidney cells. In some embodiments, the cells also express CaSR, either endogenously, or have been engineered to express CaSR. In some embodiments, cells expressing the protein (or extracts or purified preparations from cells) are contacted with a test compound to observe stimulation or inhibition of a functional response. In some embodiments, the expression level of GPR64 is assayed. In some embodiments, the test compound is assayed for its ability to antagonize CaSR-mediated inactivation of adenylate cyclase, for its ability to increase or decrease cAMP levels, and/or for its ability to enhance or inhibit parathyroid hormone secretion.

In some embodiments, the assays test binding of a candidate compound to the GPR64 or assays involving competition with a labeled competitor. In some embodiments, inhibitors can be tested in the presence of an agonist and the effect on activation by the agonist in the presence of the candidate compound is observed.

Examples of agonists or inhibitors include nucleic acids, antibodies, peptides, carbohydrates, or small molecules which bind to the protein. These agents can be selected and screened 1) at random, 2) by a rational selection or 3) by design using for example, ligand modeling techniques (e.g., computer modeling).

For random screening, agents such as antibodies, peptides, carbohydrates, small molecules and the like are selected at random and are assayed.

In some embodiments, agents can be rationally selected or designed. As used herein, an agent is said to be “rationally selected or designed” when the agent is chosen based on the configuration of the GPR64 or its target transcripts. For example, antibodies can be raised against one or more GPR64 epitopes.

In one aspect, the invention provides a method of screening for an agent which modulates the activity of GPR64, e.g., an agonist or inhibitor, comprising: (a) contacting cells expressing GPR64 with the agent to be tested; and (b) assaying the agent's effect on the expression level or activity of GPR64. In some embodiments, the activity to be tested is increased or decreased parathyroid hormone secretion, and/or increased or decreased levels of cAMP production.

In some embodiments, an inhibitor will lead to decreased levels of cAMP production over control cells. In some embodiments, an agonist will increase the levels of cAMP production over control cells. In some embodiments, cells expressing GPR64 and CaSR are administered high levels of calcium, and the ability of the inhibitor or agonist to modulate the levels of cAMP are assayed. In some embodiments, cells are stimulated with about 3 mM Ca′ for 30 min. In some embodiments, cAMP production can be assayed by measuring the level of a reporter gene that is responsive to cAMP levels. In some embodiments, the reporter gene luciferase is located downstream of a cAMP response element. In some embodiments, cAMP can be measured using a cAMP ELISA kit (Enzo Life Sciences# ADI-900-163).

In some embodiments, an inhibitor will lead to decreased parathyroid hormone secretion over control cells. In some embodiments, an agonist will lead to increased parathyroid hormone secretion over control cells. In some embodiments, parathyroid hormone secretion can be measured in a colorimetric ELISA assay (Immutopics International #60-3000).

In one embodiment, the present invention is directed to methods of screening for agents that inhibit GPR64. In some embodiments, the method comprises treating a cell, such as a parathyroid glandular cell, with a candidate agent, and detecting whether a level or activity of GPR64 is reduced, thereby screening the candidate agent for anti-GPR64 activity. In some embodiments, the present invention relates to a method of screening for an inhibitor which inhibits the activity of GPR64 comprising: (a) contacting a cell expressing GPR64 with an agent to be tested; and (b) assaying expression levels of GPR64. In some embodiments, mRNA levels (or cDNA) are assayed. In some embodiments, protein levels are assayed.

While the invention has been described with reference to certain particular examples and embodiments herein, those skilled in the art will appreciate that various examples and embodiments can be combined for the purpose of complying with all relevant patent laws (e.g., methods described in specific examples can be used to describe particular aspects of the invention and its operation even though such are not explicitly set forth in reference thereto).

The present invention is further illustrated by the following Examples. These Examples are provided to aid in the understanding of the invention and are not to be construed as a limitation thereof.

### EXAMPLES

### Example 1—Orphan Adhesion GPCR, GPR64 is Overexpressed in Parathyroid Tumors and Inhibits Calcium-Sensing Receptor-Mediated Signaling

### Materials and Methods

### Reagents and Antibodies

Reagents and antibodies were purchased from the following companies: Forskolin (Sigma# F6886), Calcium chloride (Sigma# 21115), H-89 (Cell Signaling Technologies# 9844), U0126 (Cell Signaling Technologies# 9903), Collagenase (Sigma# C2674), Zeocin (Thermo Fisher Scientific# R25001), Poly-D-Lysine (Sigma# 6407), mouse anti-FLAG (Cell Signaling Technologies# 8146), rabbit anti-N-terminal epitope of GPR64 (Atlas Antibodies AB# HPA001478), mouse anti-CaSR antibody (Thermo Fisher Scientific# MA1-934), HRP-linked horse anti-mouse IgG (Cell Signaling Technologies#7076).

### Peptide Synthesis

A 15-amino acid long peptide corresponding to amino acids 607-621 of human GPR64 was synthesized by GenScript using the solid phase peptide synthesis (SPPS) method followed by deprotecting via Fmoc chemistry. Acetonitrile, water and TFA were used for peptide purification leading to >95% purity using a reverse-phase HPLC approach. The peptide was analyzed by HPLC and mass spectrometry to confirm the correct [M+H]⁺ and was dissolved in DMSO and stored at −80° C.

### Human Subjects

Human parathyroid tissue samples (adenoma and ipsilateral normal gland biopsies) were collected and de-identified according to an Institutional Review Board-approved protocol from consented patients undergoing surgery at The University of Maryland School of Medicine. Deidentified samples were examined histologically to confirm parathyroid identity.

### Parathyroid Cell Isolation and Stimulation

Dispersed primary human parathyroid cells were rested for 1 hr at 37° C. in keratinocyte-SFM media (Thermo Fisher Scientific#37010-022) supplemented with the manufacturer's provided media supplements, antibiotics and 1.25 mM Ca²⁺. Cells were then centrifuged and transferred to KSFM media with various concentrations of Ca²⁺, P-15 peptide, forskolin and volumetric equivalent of DMSO for 30 min at 37° C. Supernatants were collected by centrifugation at 10,000 rpm, 5 min at 4° C. and were stored in −80 for determination of intact PTH.

### PTH Measurement

Human intact PTH (1-84) in cell supernatants was measured in a colorimetric ELISA assay (Immutopics International #60-3000).

### Cell Culture, Generation of Stable Cell Line and Transient Transfection

AD-293 (HEK) cells were purchased from Agilent Technologies (#240085) and cultured in DMEM media (Sigma# D6429) supplemented with 10% FBS (Sigma#12303C), 100 U/ml penicillin and 100 μg/ml streptomycin (Thermo Fisher Scientific#15140-122). HEK cells were transfected with pcDNA3.1 plasmid encoding the FLAG-tagged CaSR and a clone stably expressing the functional receptor on the surface (HEK-CaSR) was selected in the presence of 0.5 mg/ml zeocin. Cells were starved in DMEM (Thermo Fisher Scientific#21068028) supplemented with glutamine and 1.25 mM Ca²⁺ overnight before the experiment. Assays were performed in the starvation media, unless otherwise mentioned. Transfection was performed with Lipofectamine 2000 transfection reagent (Thermo Fisher Scientific#11668019).

### Gene Cloning and Mutagenesis

The pcDNA3.1 plasmid encoding the human CaSR variant 1 tagged with FLAG peptide inserted between amino acids 371 and 372 (pcDNA3.1-FLAG-CaSR) was generated as previously described (Koh J, Dar M, Untch B R, Dixit D, Shi Y, Yang Z, et al. Regulator of G protein signaling 5 is highly expressed in parathyroid tumors and inhibits signaling by the calcium-sensing receptor. Mol Endocrinol. 2011; 25(5):867-76.). pCRE-Luc plasmid was kindly provided by Evi Kostenis (University of Bonn). The human GPR64 variant 1 (full length with 1017 amino acids) was amplified from PFN21A-Halo plasmid (Promega# FHC11075) by using primers (for: TTTAAACTTAAGGCCATGGTTTTCTCTGTCAGGCA (SEQ ID NO:4) and rev: CGAGCGGCCGCTTACATTTGCTCAATAAAGTGTAA) (SEQ ID NO:5) and then inserted into pcDNA3.1 plasmid at restriction sites AflII and NotI (pcDNA3.1-GPR64). Mutations were generated by using Q5 Site-Directed Mutagenesis Kit (New England Biolabs# E0552S); To construct pcDNA3.1-GPR64ΔNTF, a GPR64 missing the N-terminal fragment (amino acids 38-606), we used pcDNA3.1-GPR64 as template and the following primers (for: ACAAGCTTCGGCGTTCTG (SEQ ID NO:6) and rev: CGATCCAGCGTAATCTGG) (SEQ ID NO:7). All constructs were verified by complete double stranded sequencing.

### Immunohistochemistry, H&E Staining and Bright-Field Imaging

The paraffin-embedded tissues were sectioned (5 μm thick) and immunostained using the Dako EnVision FLEX+Detection system (DAKO# K8000). Antigen retrieval was performed by heating at low pH for 20 min and section were rinsed in Dako wash buffer according to the manufacturer's instructions. Endogenous peroxidase activity was blocked with Peroxidase-Blocking Reagent (10 min) before incubation with rabbit anti-GPR64 antibody at 1:400 for 20 min at RT. The primary antibody signal was amplified by EnVision FLEX+Rabbit (LINKER) before incubation with EnVision FLEX/HRP Detection Reagent for 30 min. Finally, sections were stained with 3,3′-Diaminobenzidine (DAB) followed by counterstaining with Dako FLEX hematoxylin. Slides were rinsed and mounted in Cytoseal XYL (Thermo Scientific, Waltham, Mass.). Hematoxylin and Eosin staining (H & E) was performed as previously described. The bright field imaging was conducted by 40× oil objective (1.4 NA) on a Nikon Ti-E microscope equipped with 16.2 MegaPixels DS-Ri2 camera. DAB-stained pixels were defined for the Nikon NIS-Elements Basic Research software and were used for calculating the stained area fraction by ROI statistics module.

### Immunocytochemistry and Fluorescence Imaging

HEK cells were seeded on glass coverslips coated with Poly-D-Lysine (50 μg/ml) and were transfected with 2 μg plasmids. After overnight starvation, cells were fixed in cold aceton/methanol (1:1 volumetric) for 20 min at −20° C. After washing in PBS, cells were incubated with 5% goat serum in PBS for 1 hr at RT as blocking step followed by overnight incubation with rabbit anti-GPR64 (1:200) antibody in 1% BSA in PBS at 4° C. Cells were then stained with AlexaFluor 594-conjugated goat anti-rabbit (1:500) for 2 hrs at RT. Freshly isolated human parathyroid cells were fixed in cold aceton/methanol (1:1 volumetric) for 20 min at −20° C. After washing in PBS, cells were incubated with 5% goat serum in PBS for 1 hr at RT as blocking step followed by overnight incubation with isotype control antibodies or rabbit anti-GPR64 (1:200)+mouse anti-CaSR antibodies (1:1000) in 1% BSA in PBS at 4° C. Cells were then stained with AlexaFluor 594-conjugated goat anti-rabbit (1:500) and AlexaFluor 488-conjugated goat anti-mouse (1:500) for 2 hrs at RT. All cells were mounted on ProLong® Diamond Antifade Mountant with DAPI (Thermo Fisher Scientific# P36971) for nuclear counterstaining. Fluorescence microscopy was conducted by 40× oil objective (1.4 NA) on a Nikon Ti-E microscope equipped with 16.2 MegaPixels DS-Ri2 camera and images were analyzed with Nikon NIS-Elements Basic Research software.

### CRE Reporter Gene Assays

HEK cells were seeded in white opaque 96-well plates (30,000 cell/well) and were transfected with pCRE-Luc (100 ng/well) along with different doses of empty or GPR64-expressing plasmids. Cell were either left untreated or stimulated with different concentrations of P-15 peptide for 5 hrs at 37° C. Luminescence was measured in a FLEXStationIII plate reader (Molecular Devices).

cAMP Accumulation Assay

HEK and HEK-CaSR cells were seeded in 6-well plates and were transfected with 2 μg of plasmids. Forty eight hours post-transfection, cell were stimulated as follows: HEK-CaSR cells were either kept at normocalcemic condition (1.25 mM Ca²⁺) or were stimulated with 3 mM Ca²⁺ for 30 min. This was followed by stimulation with either DMSO (vehicle), forskolin (10 μM) or P-15 peptide (100 μM) for an additional 30 min in media containing either 1.25 or 3 mM Ca²⁺. HCL (0.1M) was used to stop the stimulation and cleared supernatants were used for cAMP measurement by cAMP complete ELISA kit (Enzo Life Sciences# ADI-900-163) and protein measurement by BCA method in a Beckman Coulter DTX880 microplate reader. Data are reported as pico mole cAMP per mg protein.

### Cell Surface Receptor ELISA

Cells were seeded in 96-well plates and 48 hrs post-transfection were fixed in 4% paraformaldehyde for 15 min. TBS buffer was used for washing followed by a 30-min blocking in TBSM (TBS+3% non-fat dry milk). Then cells were incubated with anti-FLAG antibody (1:3000) in TBSB (TBS+3% BSA) for 2 hrs at RT followed by a 1-hr incubation with HRP-conjugated goat anti-mouse antibody (1:3000) in TBSM. After washing 5 times with TBS, cells were incubated with TMB (Sigma# t0440) for 5 min at RT. Reaction was stopped by using the same volume of 1 N HCl and absorbance was measured at 450 nm in Beckman Coulter DTX880 microplate reader. Data were normalized to the values of pcDNA3.1 transfected cells.

### Data Analysis

Statistical analyses were conducted using appropriate tests for comparisons between two or multiple groups using GraphPad Prism 6.05 (GraphPad, San Diego, Calif., USA); P<0.05 was considered to be significant.

### Results

### GPR64 is Expressed in Parathyroid of Healthy Subjects and is Upregulated in Parathyroid Adenomas of PHPT Patients

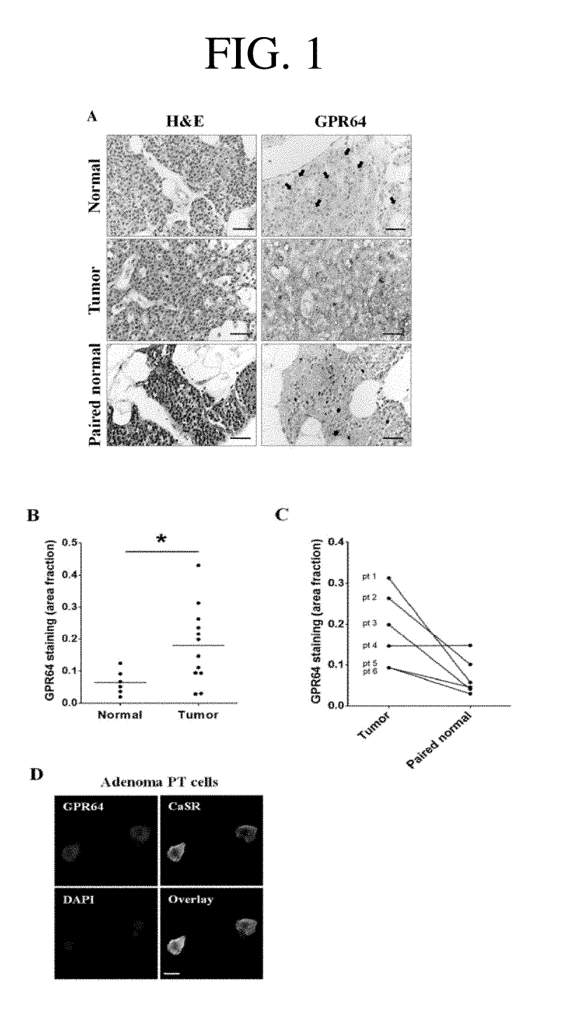

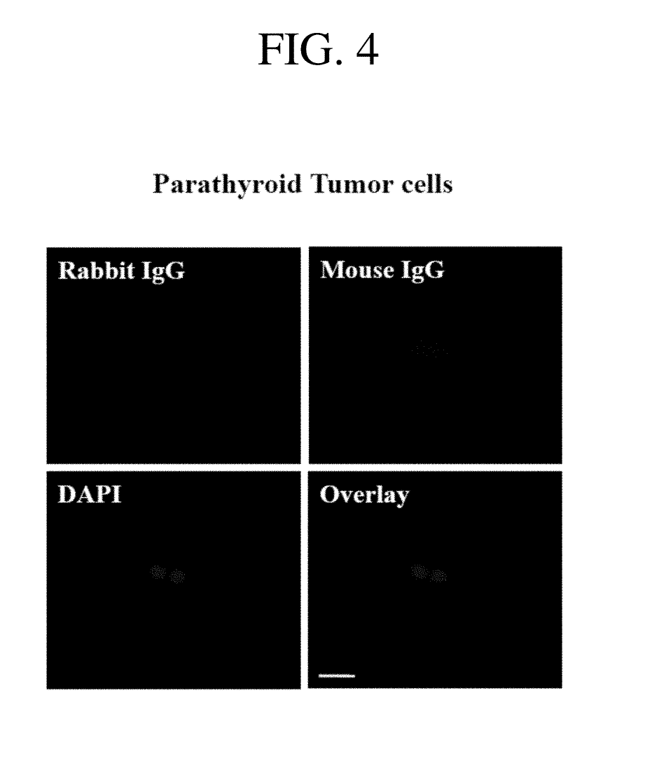

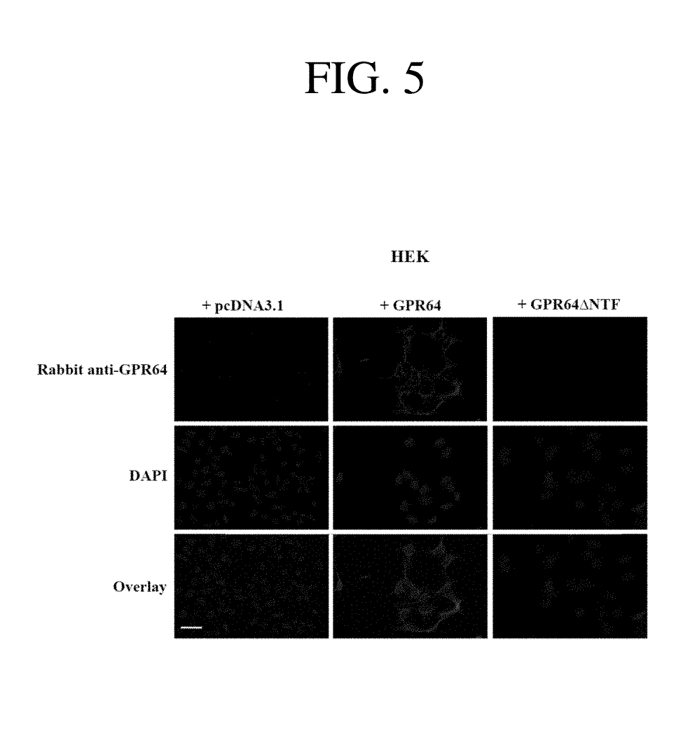

It was previously reported a comparative transcriptome analysis of genes expressed in parathyroid (adenomas, hyperplasias, and normal glands) against a panel of tumor cell lines from different origins to reveal parathyroid-specific genes. Koh et al., Mol Endocrinol. 2011; 25(5):867-76. Among many genes known to mediate parathyroid biology including PTH, VDR, and CASR, we identified GPR64 as a gene highly expressed in parathyroid tumors. Koh et al., Mol Endocrinol. 2011; 25(5):867-76. Array expression data was confirmed by in silico analysis of publicly available data (GenBank entry# AA782155.1 and The Human Protein Atlas# ENSG00000173698). It was independently confirmed parathyroid tissue expression of GPR64 and it was evaluated for pathologic expression by immunohistochemical analysis. Specific staining of GPR64 was seen on the surface of parathyroid cells at the cell-cell junctions in normal glands from cadaveric donors (FIG. 1A and Supporting FIGS. 1A and B). Consistent with the array data, expression of GPR64 was found to be highly upregulated in PHPT adenomas compared to normal parathyroids (FIGS. 1A and 1B). However, normal glands from PHPT patients did not show an upregulation of GPR64 (FIGS. 1A and 1C), suggesting that such differential expression may not be due to dysregulated Ca²⁺/PTH balance but rather an intrinsic phenotype of adenomatous gland. Immunofluorescence staining in dispersed parathyroid adenoma cells showed coexpression of CaSR and GPR64 (FIG. 1D), after confirming the specificity of GPR64 antibody in HEK cells overexpressing either full length GPR64 or its mutant lacking the antibody binding epitope in the N-terminal segment (FIGS. 4 and 5).

GPR64 Activates the cAMP-PKA-CREB Pathway in HEK Cells

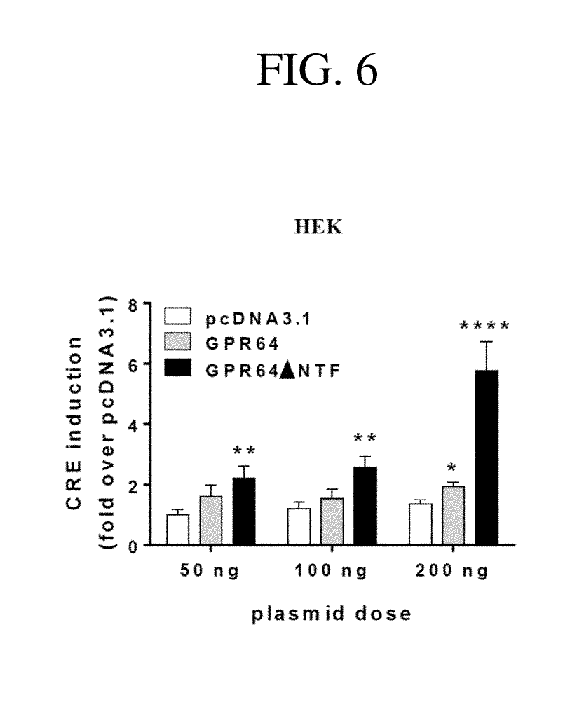

To investigate the signaling cascades emanating from GPR64, HEK cells were transfected with full length “human” GPR64 or a constitutively active mutant of GPR64 that lacks the amino acids N-terminal to the GPCR proteolysis site (GPS) (GPR64ΔNTF). Demberg et al., Biochem Biophys Res Commun. 2015; 464(3):743-7. Overexpression of full length GPR64 led to a modest induction of cAMP response element (CRE) compared to empty vector (FIG. 6). Moreover, the GPR64ΔNTF mutant showed a significantly higher CRE induction compared to full length GPR64 or empty vector in a dose-dependent manner (FIG. 6).

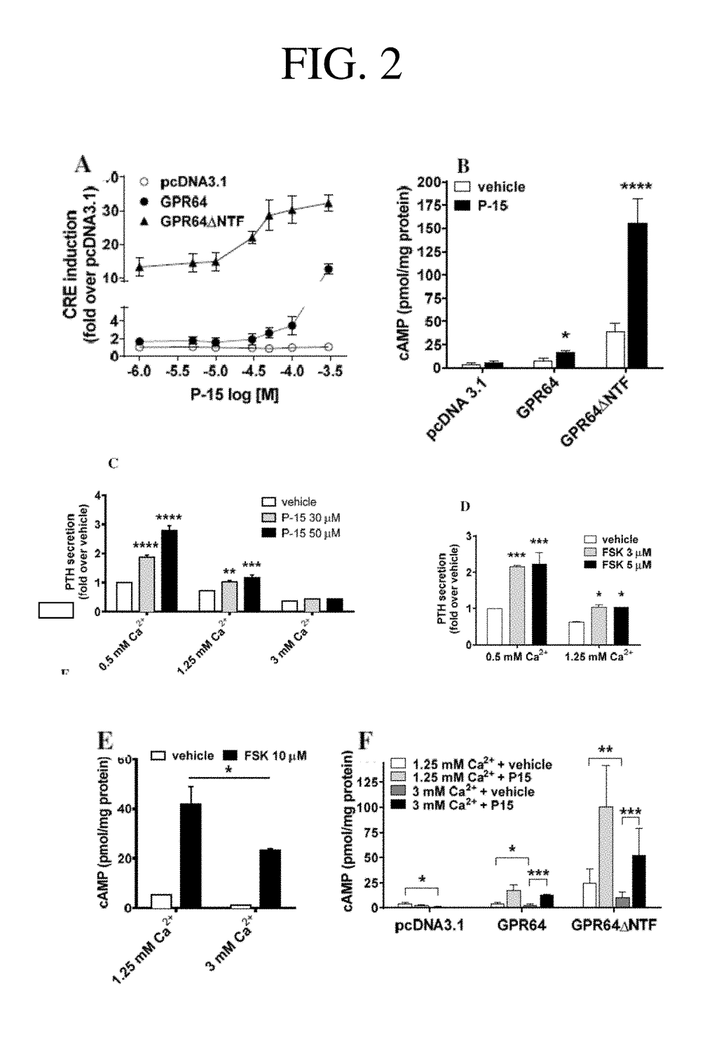

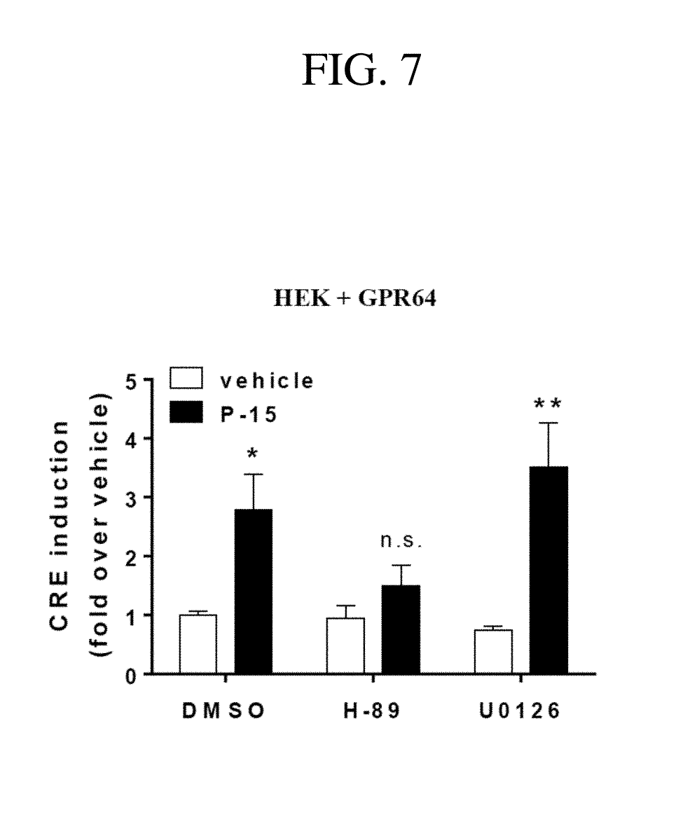

The extracellular sequence C-terminal to the GPS site (stachel sequence) has been previously shown to activate several adhesion GPCRs including “mouse” GPR64. Demberg et al., Biochem Biophys Res Commun. 2015; 464(3):743-7. A 15-aa stachel sequence of “human” GPR64 (P-15) was synthesized and a concentration-dependent CRE induction was observed in response to P-15 in GPR64- and GPR64ΔNTF-transfected cells that was absent in control cells (FIG. 2A). Various pathways can culminate in CRE induction including protein kinase A (PKA) and ERK1/2 MAPK enzymes. Whereas a specific inhibitor of PKA (H-89) blunted the P-15-induced CRE induction, the MAP kinase inhibitor (U0126) did not inhibit CRE induction (FIG. 7). The immediate second messenger of the Gas pathway, cAMP, upstream of PKA was then investigated. Consistent with CRE assay, it was also found that P-15 specifically activates GPR64 to produce cAMP and the ΔNTF mutant shows significantly higher cAMP production in both basal and P-15-activated states (FIG. 2B). Together, these results confirm that human P-15 peptide specifically activates human GPR64 to initiate a Gas-cAMP-PKA-CRE cascade and the N-terminal fragment of GPR64 may act as an intrinsic inhibitor of receptor signaling under normal conditions.

Activation of GPR64 Elevates PTH Release from Tumor Parathyroid Cells

To examine the role of GPR64 in parathyroid cell function, freshly dispersed human parathyroid adenoma cells were treated with various concentrations of Ca²⁺ and P-15. Calcium stimulation of CaSR suppressed PTH secretion in a dose-dependent manner. Interestingly, concomitant activation of GPR64 by P-15 elevated the PTH secretion (FIG. 2C).

### GPR64 Antagonizes CaSR Signaling Via Activating Adenylate Cyclase

Consistent with previous studies, accumulation of cAMP by adenylate cyclase after forskolin treatment augmented PTH secretion in parathyroid cells (FIG. 2D). Brown et al., Endocrinology. 1978; 103 (6): 2323-33. Furthermore, while forskolin increased the cAMP level in HEK-CaSR cells kept at normocalcemic condition (1.25 mM Ca²⁺), activation of CaSR by 3 mM Ca²⁺ suppressed cAMP levels significantly (FIG. 2E). These results confirm the coupling of CaSR to Gαi protein and the consequent negative impact on adenylate cyclase activity and PTH secretion.

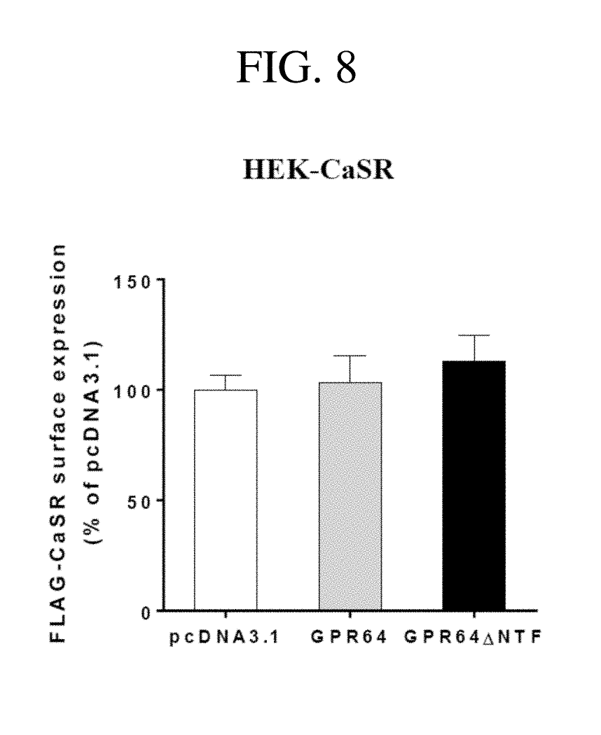

To examine mechanisms by which GPR64 elevates the PTH secretion in parathyroid cells, it was sought to identify a possible crosstalk between GPR64 and CaSR in a HEK-CaSR recombinant system. Activation of GPR64 by P-15 increased cAMP production irrespective of CaSR activation with 3 mM Ca²⁺ (FIG. 2F). GPR64ΔNTF increased the cAMP level due to its constitutive activity and although activation of CaSR with 3 mM Ca²⁺ reduced this effect, the cAMP level was still higher than that of cell transfected with empty vector in normocalcemic conditions. In addition, concomitant activation of GPR64ΔNTF-transfected cells with 3 mM Ca²⁺ and P-15 peptide restored the intracellular cAMP to the level of cells activated only with P-15 in normocalcemic conditions (FIG. 2F). A change in surface expression of CaSR upon co-expression of either GPR64 or GPR64ΔNTF (FIG. 8) was not observed. These results suggest a role for GPR64 in parathyroid adenomas where its activation can oppose the CaSR-mediated adenylate cyclase inactivation and the consequent dampening of cAMP and suppression of PTH secretion.

1-33. (canceled)

34. A method of screening for an agent which modulates the expression level or activity of GPR64 comprising:
i) contacting cells expressing GPR64 with the agent; and
ii) assaying the agent's effect on the expression level or activity of GPR64.

35. The method of claim 34, wherein the activity of GPR64 is selected from the group consisting of the level of parathyroid hormone secretion, the level of cAMP production and a combination thereof.

36. The method of claim 34, wherein the cells have been engineered to express GPR64.

37. The method of claim 34, wherein the cells express CaSR.

38. The method of claim 34, wherein the cells have been engineered to express CaSR.

39. The method of claim 34, wherein the cells are parathyroid cells.

40. The method of claim 34, wherein the cells are human embryonic kidney (HEK) cells.

41. The method of claim 34, wherein the expression level of GPR64 is assayed.

42. The method of claim 34, wherein the level of GPR64 mRNA is assayed.

43. The method of claim 34, wherein the level of GPR64 protein is assayed.

44. The method of claim 34, wherein the level of parathyroid hormone secretion is assayed.

45. The method of claim 34, wherein the level of cAMP production is assayed.

46. The method of claim 34, wherein the cells express a reporter gene downstream from a cAMP responsive element.

47. The method of claim 34, wherein the agent to be tested is an inhibitor and which reduces parathyroid hormone secretion.

48. The method of claim 34, wherein the agent to be tested is an inhibitor and which reduces the level of cAMP production.

49. The method of claim 34, wherein the agent is a nucleic acid.

50. The method of claim 34, wherein the agent is an antibody.

51. The method of claim 34, wherein the agent to be tested is an agonist and which increases parathyroid hormone secretion.

52. The method of claim 34, wherein the agent to be tested is an agonist and which increases the level of cAMP production.

53-62. (canceled)

63. A method of increasing secretion of parathyroid hormone in a subject, comprising administering to the subject a therapeutically effective amount of an agonist or activator of GPR64.

64-65. (canceled)

66. The method of claim 63, wherein the subject is administered an effective amount of a combination of an agonist or activator of GPR64 and a vector encoding GPR64 or a biologically active fragment or derivative thereof.
